(19) United States
(12) Patent Application Publication
(10) Pub. No.: US 2010/0240617 A1
(43) Pub. Date: Sep. 23, 2010

(54) BICYCLIC SPHINGOSINE 1-PHOSPHATE ANALOGS

(75) Inventors: Kevin R. Lynch, Charlottesville, VA (US); Timothy L. MacDonald, Charlottesville, VA (US); Kevin Guckian, Marlborough, MA (US); Edward Yin-shiang Lin, Chestnut Hill, MA (US); Bin Ma, Arlington, MA (US)

Correspondence Address: Prout and Associates, LLC, P.O. Box 761, Wayzata, MN, 55391, US

(73) Assignees: University of Virginia Patent Foundation; Biogen Idec MA Inc.

(21) Appl. No.: 12/705,969

(22) Filed: Feb. 15, 2010

Related U.S. Application Data
(63) Continuation of application No. PCT/US2008/073378, filed on Aug. 15, 2008.
(60) Provisional application No. 60/956,111, filed on Aug. 15, 2007.

(51) Int. Cl. A61K 31/661; C07C 317/10; C07C 49/215; C07C 229/34; C07C 211/38; C07F 9/09; C07D 271/06; C07D 271/10; C07D 333/02; A61K 31/122; A61K 31/235; A61K 31/135; A61K 31/4245; A61K 31/381; A61P 37/02

(52) U.S. Cl. 514/143; 568/31; 568/328; 560/38; 564/428; 558/158; 558/166; 548/131; 548/143; 549/78; 514/675; 514/532; 514/653; 514/364; 514/438

(57) ABSTRACT

Compounds that have agonist activity at one or more of the S1P receptors are provided. The compounds are sphingosine analogs that, after phosphorylation, can behave as agonists at S1P receptors.

### CROSS-REFERENCE TO RELATED APPLICATIONS

This application is a continuation of International Application No. PCT/US2008/073378, filed on Aug. 15, 2008, which claims priority to Provisional Application No. 60/956,111, filed Aug. 15, 2007, the disclosures of all of which are incorporated by reference in their entirety.

### US GOVERNMENT RIGHTS

This invention was made with United States Government support under Grant No. RO1 GM 067958 awarded by the National Institutes of Health. The United States Government has certain rights in the invention.

### BACKGROUND OF THE INVENTION

Sphingosine 1-phosphate (S1P) is a lysophospholipid mediator that evokes a variety of cellular responses by stimulation of five members of the endothelial cell differentiation gene (EDG) receptor family. The EDG receptors are G-protein coupled receptors (GPCRs) and on stimulation propagate second messenger signals via activation of heterotrimeric G-protein alpha (G_{α}) subunits and beta-gamma (G_{βγ}) dimers. Ultimately, this S1P-driven signaling results in cell survival, increased cell migration and, often, mitogenesis. The recent development of agonists targeting S1P receptors has provided insight regarding the role of this signaling system in physiologic homeostasis. For example, the immunomodulator, FTY-720 (2-amino-2-[2-(4-octylphenyl)ethyl]propane 1,3-diol), that following phosphorylation, is an agonist at 4 of 5 S1P receptors, revealed that enhancing S1P tone influences lymphocyte trafficking. Further, S1P type 1 receptor (S1P₁) antagonists cause leakage of the lung capillary endothelium, which suggests that S1P may be involved in maintaining the integrity of the endothelial barrier in some tissue beds.

Sphingosine 1-phosphate (S1P) is a lysophospholipid mediator that evokes a variety of cellular responses by stimulation of five members of the endothelial cell differentiation gene (EDG) receptor family.

Sphingosine-1-phosphate (S1P) has been demonstrated to induce many cellular processes, including those that result in platelet aggregation, cell proliferation, cell morphology, tumor-cell invasion, endothelial cell chemotaxis and angiogenesis. For these reasons, S1P receptors are good targets for therapeutic applications such as wound healing and tumor growth inhibition.

Sphingosine-1-phosphate signals cells in part via a set of G protein-coupled receptors named S1P₁, S1P₂, S1P₃, S1P₄, and S1P₅ (formerly EDG1, EDG5, EDG3, EDGE and EDGE). The EDG receptors are G-protein coupled receptors (GPCRs) and on stimulation propagate second messenger signals via activation of heterotrimeric G-protein alpha (G_{α}) subunits and beta-gamma (G_{βγ}) dimers. These receptors share 50-55% amino acid sequence identity and cluster with three other receptors (LPA₁, LPA₂, and LPA₃ (formerly EDG2, EDG4 and EDG7) for the structurally related lysophosphatidic acid (LPA).

A conformational shift is induced in the G-Protein Coupled Receptor (GPCR) when the ligand binds to that receptor, causing GDP to be replaced by GTP on the α-subunit of the associated G-proteins and subsequent release of the G-proteins into the cytoplasm. The α-subunit then dissociates from the βγ-subunit and each subunit can then associate with effector proteins, which activate second messengers leading to a cellular response. Eventually the GTP on the G-proteins is hydrolyzed to GDP and the subunits of the G-proteins re-associate with each other and then with the receptor. Amplification plays a major role in the general GPCR pathway. The binding of one ligand to one receptor leads to the activation of many G-proteins, each capable of associating with many effector proteins leading to an amplified cellular response.

S1P receptors make good drug targets because individual receptors are both tissue and response specific. Tissue specificity of the S1P receptors is desirable because development of an agonist or antagonist selective for one receptor localizes the cellular response to tissues containing that receptor, limiting unwanted side effects. Response specificity of the S1P receptors is also of importance because it allows for the development of agonists or antagonists that initiate or suppress certain cellular responses without affecting other responses. For example, the response specificity of the S1P receptors could allow for an S1P mimetic that initiates platelet aggregation without affecting cell morphology.

Sphingosine-1-phosphate is formed as a metabolite of sphingosine in its reaction with sphingosine kinase and is stored in abundance in the aggregates of platelets where high levels of sphingosine kinase exist and sphingosine lyase is lacking S1P is released during platelet aggregation, accumulates in serum, and is also found in malignant ascites. Reversible biodegradation of S1P most likely proceeds via hydrolysis by ectophosphohydrolases, specifically the sphingosine 1-phosphate phosphohydrolases. Irreversible degradation of S1P is catalyzed by S1P lyase yielding ethanolamine phosphate and hexadecenal.

Currently, there is a need for novel, potent, and selective agents that are agonists of the S1P receptor having enhanced potency, selectivity, and oral bioavailability. In addition, there is a need in the art for identification of, as well as the synthesis and use of, such compounds. The present invention satisfies these needs.

### SUMMARY

The present invention provides in one aspect specific stereoisomers of compounds having agonist activity at one or more of the S1P receptors. The compounds are sphingosine analogs that, after phosphorylation, can behave as agonists at S1P receptors. Accordingly, there is provided enantiomers of compounds of formula I:

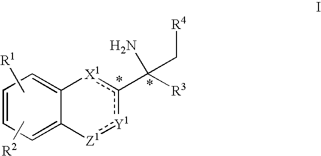

wherein X¹, Y¹ and Z¹ are independently O, CR^{a}, CR^{a}R^{b}, N, NR^{c}, or S; R¹ and R² are independently hydrogen, halo, halo(C₁-C₁₀)alkyl, cyano, —NR^{a}R^{b}, (C₁-C₂₀)alkyl, (C₂-C₂₀)alkenyl, (C₂-C₂₀)alkynyl, (C₁-C₂₀)alkoxy, (C₂-C₂₆)alkoxyalkyl, (C₃-C₁₂)cycloalkyl, (C₆-C₁₀)aryl, (C₇-C₃₀)arylalkyl, (C₂-C₁₀)heterocyclic, (C₄-C₁₀)heteroaryl, or (C₄-C₁₀)-heteroaryl(C₁-C₂₀)alkyl; or
R² can be a group having formula II, III, IV, V, or VI:

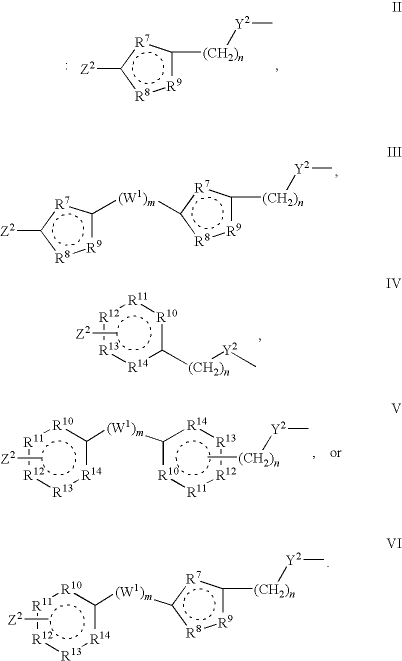

R⁷, R⁸, R⁹, R¹⁰, R¹¹, R¹², R¹³, and R¹⁴ are independently O, S, C, CR¹⁵, CR¹⁶R¹⁷, C═O, N or NR¹⁸; R¹⁵, R¹⁶ and R¹⁷ are independently hydrogen, halo, (C₁-C₁₀)alkyl, (C₁-C₁₀)alkyl substituted with halo, hydroxy, (C₁-C₁₀)alkoxy, or cyano; and where R¹⁸ can be hydrogen or (C₁-C₁₀)alkyl;
Z² is hydrogen, halo, halo(C₁-C₁₀)alkyl, cyano, —NR^{a}R^{b}, (C₁-C₂₀)alkyl, (C₂-C₂₀)alkenyl, (C₂-C₂₀)alkynyl, (C₁-C₂₀)alkoxy, (C₂-C₂₆)alkoxyalkyl, (C₃-C₁₂)cycloalkyl, (C₆-C₁₀)aryl, (C₇-C₃₀)arylalkyl, (C₂-C₁₀)heterocyclic, (C₄-C₁₀)heteroaryl, or (C₄-C₁₀)heteroaryl(C₁-C₂₀)alkyl. The alkyl, alkenyl, alkynyl, cycloalkyl, aryl, heterocyclic, or heteroaryl groups of Z² are optionally perfluorinated or optionally substituted with 1, 2, 3, or 4 groups where the substituent groups are independently hydroxy, halo, cyano, (C₁-C₁₀)alkoxy, C₆-aryl, (C₇-C₂₄)arylalkyl, oxo (═O), or imino (═NR^{d}), wherein one or more of the carbon atoms in the Z² alkyl groups can be independently replaced with non-peroxide oxygen, sulfur or NR^{c}; [symbol] indicates one or more optional double bonds; Y² is a bond (absent), O, S, C═O, or NR^{c}, CH₂; W¹ is a bond; —CH₂— and m is 1, 2, or 3, or (C═O)(CH₂)₁₋₅ and m is 1; wherein W¹ is optionally interrupted with non-peroxide O, S, C═O, or NR^{c}. Each [symbol] represents an optional double bond; and n is 0, 1, 2, or 3.

R³ is hydrogen, (C₁-C₁₀)alkyl, hydroxy(C₁-C₁₀)alkyl or (C₁-C₁₀)alkoxy; and R⁴ is hydroxyl (—OH), phosphate (—OPO₃H₂), phosphonate (—CH₂PO₃H₂), or alpha-substituted phosphonate; and R^{a}, R^{b}, and R^{c} are independently hydrogen, or (C₁-C₁₀)alkyl.

The alkyl, alkenyl, alkynyl, cycloalkyl, aryl, heterocyclic, or heteroaryl groups of R¹ and R² independently are optionally perfluorinated or optionally substituted with 1, 2, 3, or 4 groups where the substituent groups are independently hydroxy, halo, cyano, (C₁-C₁₀)alkoxy, C₆-aryl, (C₇-C₂₄)arylalkyl, oxo (═O), or imino (═NR^{d}), wherein one or more of the carbon atoms in the R¹ or R² alkyl groups can be independently replaced with non-peroxide oxygen, sulfur or NR^{c}. The alkyl groups of R³ are optionally substituted with 1, or 2 hydroxy groups; and R^{d} is hydrogen, or (C₁-C₁₀)alkyl. The compounds are enantiomers of the compounds of formula I. The invention includes pharmaceutically acceptable salts or esters of the compounds of formula I.

The compounds of formula I are enantiomerically pure and can have one or two chiral centers. The chiral carbon centers are indicated by “*” in formula I:

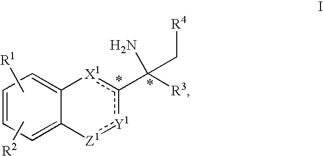

except when [symbol] indicates a double bond at the carbon. The compounds having one chiral carbon can have either an R or S configuration. The compounds having two chiral carbons can have R,R, R,S, S,R, or S,S configurations, where the first letter indicates the configuration of the ring carbon and the second refers to the configuration of the carbon in the chain.

In another aspect, the invention provides phosphate esters having formula VII.

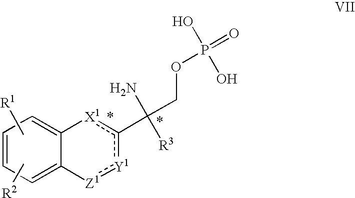

wherein R¹, R², R³, X¹, Y¹, and Z¹ are as described above. In another aspect, the invention provides enantiomers of the compounds having formula I or Formula VII have the RR, RS, SR or SS configuration.

In another aspect, the invention provides enantiomeric pro-drugs of the compounds of formula I. In another aspect, the invention also provides enantiomeric compounds of formula I, formula IV or pharmaceutically acceptable salts or esters thereof for use in medical therapy.

In another aspect, the present invention provides a method for inhibiting angiogenesis in a tumor, comprising contacting the cancerous cells with an effective amount of an enantiomeric compound of formula I, formula IV or a pharmaceutically acceptable salt thereof.

In another aspect, the invention provides a method for modulating the immune system by altering lymphocyte trafficking for treatment of autoimmune diseases or prolongation of allograft transplant survival, said method comprising administering an effective amount of at least one enantiomeric compound of formula Ito a subject in need thereof.

In another aspect, the invention provides a method for to preventing, inhibiting or treating neuropathic pain, wherein the method comprises administering an effective amount of at least one enantiomeric compound of formula I or a compound of formula I and a pharmaceutically-acceptable carrier is administered to a subject in need thereof. Pain can be nociceptive or neuropathic in nature. Neuropathic pain is characterized by its chronic nature, an absence of an obvious, direct cause (e.g., tissue damage), hyperalgesia or allodynia. Hyperalgesia is an exaggerated response to a painful stimulus. Allodynia is the perception of normal stimuli as painful (examples include the touch of clothing, warm or cool air, etc.). Neuropathic pain can be a sequel to nerve damage in an extremity such as an arm, or more often a leg. Precipitating events can include trauma, e.g., motor vehicle accidents or amputations (e.g., phantom limb pain). Neuropathic pain can occur due to an adverse effect of drug therapies, e.g., vincristine or paclitaxel (TAXOL™) or can occur as a component of disease pathologies, such as diabetes type 1 or type 2, shingles, HIV-1 infections, etc. Typically, neuropathic pain is not responsive to opiates or non-steroidal anti-inflammatory drugs such as aspirin.

In another aspect, the invention provides a method for repairing vascular injury following catheterization, comprising contacting the lumen of the affected vessel with an effective amount of the enantiomeric compound of formula I. In another aspect, the invention includes coating indwelling stents with an enantiomeric compound of formula I.

In another aspect, the present invention provides compositions and methods for the use of S1P analogs to prevent and inhibit vascular restenosis following vascular injury. For example, the injury can be due to balloon angioplasty. In another aspect, the present invention includes a method for treating subjects to prevent vascular restenosis.

In another aspect, the present invention provides compositions and methods for the use of sphingosine analogs (including S1P pro-drugs) to prevent asthma attacks. In one aspect, the asthma could be due to over production of cysteinyl leukotrienes. In another aspect, the present invention includes a method for treating subjects to treat asthma.

In another aspect, the present invention provides compositions and methods for the use of sphingosine analogs of formula I (including S1P pro-drugs) to treat obesity.

In another aspect, the present invention provides compositions and methods for the use of sphingosine analogs (including S1P pro-drugs) to normalize blood lipid composition. In one aspect, blood low density lipoprotein (LDL or ‘bad cholesterol’) levels could be lowered. In another aspect, blood triglyceride levels could be lowered.

In another aspect, the present invention provides compositions and methods for the use of S1P analogs and S1P pro-drugs for the prevention and treatment of arteriosclerosis.

In another aspect, the present invention provides compositions and methods for the use of S1P analogs and S1P pro-drugs for the treatment of neoplastic disease. In one aspect, this treatment is effected by application of S1P receptor antagonists that are efficacious by virtue of their anti-angiogenic properties. In another aspect, the treatment is effected by administration of the enantiomeric sphingosine analogs of formula I that inhibit the multiple substrate lipid kinase.

In another aspect, the present invention provides compositions and methods for the use of S1P analogs and S1P pro-drugs for the treatment of neurodegenerative diseases. In one aspect, the treatment is for senile dementia of the Alzheimers type.

In another aspect, the invention provides a compound of formula I, or a pharmaceutically acceptable salt thereof for use in medical treatment (for example, treatment of neoplastic disease, treatment of neuropathic pain, treatment of autoimmune disease, or prolongation of allograft survival).

In another aspect, the invention provides a method for the use of an enantiomeric compound of formula I or a pharmaceutically acceptable salt thereof to prepare a medicament for inhibiting tumor growth, metastasis or tumor angiogenesis in a mammalian species (for example, a human).

In another aspect, the invention provides for the use of a compound of formula I or a pharmaceutically acceptable salt thereof to prepare a medicament for treating an autoimmune disease or prolonging allograft survival in a mammalian species (for example, a human).

In another aspect, the invention provides for the use of a compound of formula I or a pharmaceutically acceptable salt thereof to prepare a medicament for treating neuropathic pain in a mammalian species (for example, a human).

In another aspect, the invention provides a method for assessing a compound of formula I (e.g., S1P receptor pro-drugs) as a substrate for sphingosine kinase types 1 or 2, in vitro and in vivo. In another aspect, the invention includes a method of assessing a compound of formula I for binding to designated receptor sites comprising in vivo or in vitro, with an amount of a compound of formula I effective to bind said receptors. Tissue comprising ligand bound designated S1P receptor sites can be used to measure the selectivity of test compounds for specific receptor subtypes, or can be used as a tool to identify potential therapeutic agents for the treatment of diseases, by contacting said agents with said ligand-receptor complexes, and measuring the extent of displacement of the ligand or binding of the agent.

In another aspect, the invention provides novel intermediates and processes disclosed herein that are useful for preparing compounds of formula I, including the generic and specific intermediates as well as the synthetic processes described herein.

In another aspect, the present invention provides synthetic schemes and methods of use of compounds having formula I and analogs or derivatives thereof. In another aspect, the invention provides synthetic and modification schemes for preparing analogs and derivatives of the compounds of formula I, as well as compositions and methods for the use of such analogs and derivatives.

In another aspect, the present invention provides synthetic schemes and methods of use of compounds having formula I and analogs or derivatives thereof. In another aspect, the invention provides pharmaceutical compositions comprising enantiomeric compounds of formula I, including, the enantiomeric compounds and a pharmaceutically acceptable carrier.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

The details of one or more embodiments of the invention are set forth in the accompanying description below. Other features, objects, and advantages of the invention will be apparent from the description and drawings, and from the claims.

### BRIEF DESCRIPTION OF THE DRAWINGS

FIGS. 1A and 1B illustrate three S1P agonists, FTY-720, AAL151, compound XXX, and additional compounds of formula I.

FIGS. 2, 3 and 4 are schemes illustrating syntheses of compounds of formula I.

FIGS. 5A, 5B and 5C are schemes illustrating syntheses of enantiomerically pure compounds of formula I.

FIG. 6 is a scheme illustrating the separation of enantiomers of formula VIII and phosphorylated compounds prepared from them.

FIG. 7 graphically illustrates the results of a broken cell GTPγ³⁵S binding assay for the human S1P₁ receptor, testing S1P and compound VIII-D.

FIG. 8 graphically illustrates the results of a calcium mobilization assay for S1P and compound VIII-D.

FIG. 9 graphically illustrates the results of a broken cell GTPγ³⁵S binding assay for the human S1P₁ receptor, testing S1P and compound VIII-C.

FIG. 10 graphically illustrates the results of a calcium mobilization assay for S1P and compound VIII-C.

FIG. 11 graphically illustrates the results of a broken cell GTPγ³⁵S binding assay for the human S1P₁ receptor, testing S1P and compound VIII-F.

FIG. 12 graphically illustrates the results of a broken cell GTPγ³⁵S binding assay for the human S1P₁ receptor, testing S1P and compounds VIII-C, VIII-F and VIII-E.

FIG. 13 graphically illustrates the results of a calcium mobilization assay for S1P and compounds FTY-720 P, VIII-F and VIII-E.

FIG. 14 graphically illustrates the results of a calcium mobilization assay for S1P and compound VIII-E.

FIG. 15 graphically illustrates the results of a broken cell GTPγ³⁵S binding assay for the human S1P₁ receptor, testing S1P and compound X-F.

FIG. 16 graphically illustrates the results of a CHO cell assay for S1P and compound X-F.

FIG. 17 graphically illustrates the results of a broken cell GTPγ³⁵S binding assay for the human S1P₁ receptor, testing S1P, FTY-720 P and compound X-E.

FIG. 18 graphically illustrates the results of a calcium mobilization assay for S1P FTY-720 P and compound X-E.

FIG. 19 graphically illustrates the results of a CHOK1 cell assay for S1P and FTY-720 P and compound X-E.

### DETAILED DESCRIPTION

The following abbreviations are used herein: S1P, sphingosine-1-phosphate; S1P₁₋₅ SIP receptor types; GPCR, G-protein coupled receptor; SAR, structure-activity relationship; EDG, endothelial cell differentiation gene; EAE, experimental autoimmune encephalomyelitis; NOD non-obese diabetic; TNFα, tumor necrosis factor alpha; HDL, high density lipoprotein; and RT-PCR, reverse transcriptase polymerase chain reaction.

In describing and claiming the invention, unless otherwise defined, all technical and scientific terms used herein have the same meaning as commonly understood by one of ordinary skill in the art to which this invention belongs. Although any materials and methods similar or equivalent to those described herein can be used in the practice or testing of the present invention, the preferred materials and methods are described herein. Each of the following terms has meaning associated with it in this section. Specific and preferred values listed below for radicals, substituents, and ranges are for illustrations only; they do not exclude other defined values or other values within defined ranges for the radicals and substituents.

The terms “a,” “an,” “the,” “at least one,” and “one or more” are used interchangeably. Thus, for example, a composition that comprises “an” element means one element or more than one element.

The term “receptor agonists” are compounds that mimic the action of S1P at one or more of its receptors but may have differing potency or efficacy.

The term “receptor antagonists” are compounds that 1) lack intrinsic agonist activity and 2) block agonist (e.g., S1P) activation of the S1P receptor(s), often in a manner that is both fully surmountable and reversible (competitive antagonist').

The term “affected cell” refers to a cell of a subject afflicted with a disease or disorder, which affected cell has an altered phenotype relative to a subject not afflicted with a disease or disorder.

Cells or tissue are “affected” by a disease or disorder if the cells or tissue have an altered phenotype relative to the same cells or tissue in a subject not afflicted with a disease or disorder.

A disease or disorder is “alleviated” if the severity of a symptom of the disease or disorder, the frequency with which such a symptom is experienced by a patient, or both, is reduced.

An “analog” of a chemical compound is a compound that, by way of example, resembles another in structure but is not necessarily an isomer (e.g., 5-fluorouracil is an analog of thymine).

The terms “cell,” “cell line,” and “cell culture” may be used interchangeably.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

The term “enantiomerically pure” refers to compounds having an enantiomeric excess (ee) of greater than about 60%. Preferably the enantiomeric excess can be greater than 80%. More preferably the enantiomeric excess can be greater than 90%. Even more preferably the enantiomeric excess can be greater than 90%. Most Preferably the enantiomeric excess can be greater than 95%.

A tissue “normally comprises” a cell if one or more of the cell are present in the tissue in an animal not afflicted with a disease or disorder.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

A “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” means an amount sufficient to produce a selected effect. For example, an effective amount of an S1P receptor antagonist is an amount that decreases the cell signaling activity of the S1P receptor.

A “functional” molecule is a molecule in a form in which it exhibits a property by which it is characterized. By way of example, a functional enzyme is one which exhibits the characteristic catalytic activity by which the enzyme is characterized.

The term “inhibit” refers to the ability of a disclosed compound to reduce or impede a described function. Preferably, inhibition is by at least 10%, more preferably by at least 25%, even more preferably by at least 50%, and most preferably, the function is inhibited by at least 75%.

“Instructional material” includes a publication, a recording, a diagram, or any other medium of expression which can be used to communicate the usefulness of the disclosed compounds in the kit for effecting alleviation of the various diseases or disorders recited herein. Optionally, or alternately, the instructional material may describe one or more methods of alleviating the diseases or disorders in a cell or a tissue of a mammal. The instructional material of the kit may, for example, be affixed to a container which contains a disclosed compound or be shipped together with a container which contains the identified compound. Alternatively, the instructional material may be shipped separately from the container with the intention that the instructional material and the compound be used cooperatively by the recipient.

The term “parenteral” means not through the alimentary canal but by some other route such as subcutaneous, intramuscular, intraspinal, or intravenous.

The term “pharmaceutically acceptable carrier” includes any of the standard pharmaceutical carriers, such as a phosphate buffered saline solution, water and emulsions such as an oil/water or water/oil emulsion, and various types of wetting agents. The term also encompasses any of the agents approved by a regulatory agency of the U.S. Federal government or listed in the U.S. Pharmacopeia for use in animals, including humans.

The term “purified” and similar terms relate to the isolation of a molecule or compound in a form that is substantially free (at least 75% free, preferably 90% free, and most preferably at least 95% free) from other components normally associated with the molecule or compound in a native environment. The term “purified” does not necessarily indicate that complete purity of the particular molecules achieved during the process. A “very pure” compound refers to a compound that is greater than 90% pure. A “highly purified” compound refers to a compound that is greater than 95% pure.

A “sample” refers preferably to a biological sample from a subject, including, but not limited to, normal tissue samples, diseased tissue samples, biopsies, blood, saliva, feces, semen, tears, and urine. A sample can also be any other source of material obtained from a subject, which contains cells, tissues, or fluid of interest. A sample can also be obtained from cell or tissue culture.

The term “standard,” refers to something used for comparison. For example, a standard can be a known standard agent or compound which is administered or added to a control sample and used for comparing results when measuring said compound in a test sample. Standard can also refer to an “internal standard,” such as an agent or compound which is added at known amounts to a sample and is useful in determining such things as purification or recovery rates when a sample is processed or subjected to purification or extraction procedures before a marker of interest is measured.

A “subject” of analysis, diagnosis, or treatment is an animal. Such animals include mammals, preferably a human.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term “treating” includes prophylaxis of the specific disorder or condition, or alleviation of the symptoms associated with a specific disorder or condition or preventing or eliminating said symptoms.

The disclosed compounds are generally named according to the IUPAC or CAS nomenclature system. Abbreviations which are well known to one of ordinary skill in the art may be used (e.g., “Ph” for phenyl, “Me” for methyl, “Et” for ethyl, “h” for hour or hours, “rt” for room temperature, “THF” for tetrahydrofuran, and “rac” for racemic mixture).

The values listed below for radicals, substituents, and ranges, are for illustration only; they do not exclude other defined values or other values within defined ranges for the radicals and substituents. The disclosed compounds include compounds of formula I having any combination of the values, specific values, more specific values, and preferred values described herein.

The term “halogen” or “halo” includes bromo, chloro, fluoro, and iodo. The term “haloalkyl”, refers to an alkyl radical bearing at least one halogen substituent, non-limiting examples include, but are not limited to, chloromethyl, fluoroethyl or trifluoromethyl and the like. The term “C₁-C₂₀ alkyl” refers to a branched or linear alkyl group having from one to twenty carbons. Non-limiting examples include, but are not limited to, methyl, ethyl, n-propyl, iso-propyl, butyl, iso-butyl, sec-butyl, tert-butyl, pentyl, hexyl, heptyl, octyl and the like. The term “C₂-C₂₀ alkenyl”, refers to an olefinically unsaturated branched or linear group having from two to twenty carbon atoms and at least one double bond. Typically, C₂-C₂₀ alkenyl groups include, but are not limited to, 1-propenyl, 2-propenyl, 1,3-butadienyl, 1-butenyl, hexenyl, pentenyl, hexenyl, heptenyl, octenyl and the like. The term (C₂-C₂₀)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl, and the like. The term “(C₁-C₁₀)alkoxy” refers to an alkyl group attached through an oxygen atom. Examples of (C₁-C₁₀)alkoxy can be methoxy, ethoxy, propoxy, isopropoxy, butoxy, iso-butoxy, sec-butoxy, pentoxy, 3-pentoxy, or hexyloxy and the like. The term “C₃-C₁₂ cycloalkyl”, can be cyclopropyl, cyclobutyl, cyclopentyl, cyclohexyl, cycloheptyl, cyclooctyl and the like.

The term “optionally substituted” refers to zero, one, two, three or four substituents, wherein the substituents are each independently selected. Each of the independently selected substituents may be the same or different than other substituents.

The term “(C₆-C₁₀)aryl” refers to a mono or bicyclic carbocyclic ring system having one or two aromatic rings including, but not limited to, phenyl, benzyl, naphthyl, tetrahydronaphthyl, indanyl, indenyl, and the like.

The term “aryl(C₁-C₂₀)alkyl” or “aralkyl” refers to an alkyl group substituted with a mono or bicyclic carbocyclic ring system having one or two aromatic rings including, a group such as phenyl, naphthyl, tetrahydronaphthyl, indanyl, indenyl, and the like. Non-limiting examples of arylalkyl include benzyl, phenylethyl, and the like.

The term “optionally substituted aryl” includes aryl compounds having zero, one, two, three or four substituents, and a substituted aryl includes aryl compounds having one, two, three or four substituents, wherein the substituents include groups such as, for example, alkyl, halo, or amino substituents.

The “(C₂-C₁₀)heterocyclic group” refers to an optionally substituted mono- or bicyclic carbocyclic ring system containing one, two, or three heteroatoms (optionally in each ring) wherein the heteroatoms are oxygen, sulfur, and nitrogen.

The term “(C₄-C₁₀)heteroaryl” refers to an optionally substituted mono- or bicyclic carbocyclic ring system containing one, two, or three heteroatoms (optionally in each ring) wherein the heteroatoms are oxygen, sulfur, and nitrogen. Non-limiting examples of heteroaryl groups include furyl, thienyl, pyridyl, and the like.

The term “bicyclic” represents either an unsaturated or saturated stable bridged or fused bicyclic carbon ring. The bicyclic ring may be attached at any carbon atom which affords a stable structure. Typically a bicyclic ring system can have from about 7- to about 12 atoms in the ring system. The term includes, but is not limited to, naphthyl, dicyclohexyl, dicyclohexenyl, and the like.

The term “phosphate analog” and “phosphonate analog” comprise analogs of phosphate and phosphonate wherein the phosphorous atom is in the +5 oxidation state and one or more of the oxygen atoms is replaced with a non-oxygen moiety, including for example, the phosphate analogs phosphorothioate, phosphorodithioate, phosphoroselenoate, phosphorodiselenoate, phosphoroanilothioate, phosphoranilidate, phosphoramidate, boronophosphates, and the like, including associated counterions, e.g., H, NH₄, Na, K, and the like if such counterions are present.

The term “alpha-substituted phosphonate” includes phosphonate (—CH₂PO₃H₂) groups that are substituted on the alpha-carbon such as —CHFPO₃H₂, —CF₂PO₃H₂, —CHOHPO₃H₂, —C═OPO₃H₂) and the like.

A “derivative” of a compound refers to a chemical compound that may be produced from another compound of similar structure in one or more steps, such as replacement of hydrogen by an alkyl, acyl, or amino group.

The term “pharmaceutically acceptable carrier” includes any of the standard pharmaceutical carriers, such as a phosphate buffered saline solution, hydroxypropyl beta-cyclodextrins (HO-propyl beta cyclodextrins), water, emulsions such as an oil/water or water/oil emulsion, and various types of wetting agents. The term also encompasses any of the agents approved by a regulatory agency of the U.S. Federal government or listed in the U.S. Pharmacopeia for use in animals, including humans.

The term “pharmaceutically-acceptable salt” refers to salts which retain the biological effectiveness and properties of the disclosed compounds and which are not biologically or otherwise undesirable. In many cases, the disclosed compounds are capable of forming acid or base salts by virtue of the presence of amino or carboxyl groups or groups similar thereto.

An “effective amount” means an amount sufficient to produce a selected effect. For example, an effective amount of an S1P receptor agonist is an amount that decreases the cell signaling activity of the S1P receptor.

The disclosed compounds can contain one or more asymmetric centers in the molecule. In accordance with the present disclosure any structure that does not designate the stereochemistry is to be understood as embracing all the various optical isomers, as well as racemic mixtures thereof.

The disclosed compounds may exist in tautomeric forms and the invention includes both mixtures and separate individual tautomers. For example, the following structure:

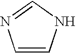

is understood to represent a mixture of the structures:

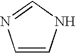

as well as

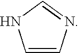

The terms 16:0, 18:0, 18:1, 20:4 or 22:6 hydrocarbon refers to a branched or straight alkyl or alkenyl group, wherein the first integer represents the total number of carbons in the group and the second integer represent the number of double bonds in the group.

An “S1P modulating agent” refers a compound or composition that is capable of inducing a detectable change in S1P receptor activity in vivo or in vitro (e.g., at least 10% increase or decrease in S1P activity as measured by a given assay such as the bioassay described in the examples and known in the art. “S1P receptor,” refers to all of the S1P receptor subtypes (for example, the S1P receptors S1P₁, S1P₂, S1P₃, S1P₄, and S1P₅), unless the specific subtype is indicated.

It will be appreciated by those skilled in the art that the disclosed compounds having chiral centers may exist in and be isolated in optically active and racemic forms. It is to be understood that the disclosed compounds encompass any racemic, optically active or stereoisomeric form, or mixtures thereof, of the compound, which possess the useful properties described herein, such as the S,R; S,S; R,R; or R,S diastereomers. It is well known in the art how to prepare such optically active forms (for example, resolution of the racemic form by recrystallization techniques, synthesis from optically-active starting materials, by chiral synthesis, or chromatographic separation using a chiral stationary phase) and how to determine S1P agonist activity using the standard tests described herein, or using other similar tests which are well known in the art. In addition, some compounds may exhibit polymorphism.

Potential uses of an SIP receptor agonist pro-drugs (S1P₁ receptor type selective agonists preferred) include, but are not limited to, altering lymphocyte trafficking as a method of treatment for autoimmune pathologies such as uveitis, type I diabetes, rheumatoid arthritis, inflammatory bowel diseases, and, most particularly, multiple sclerosis. “Treatment” of multiple sclerosis includes the various forms of the disease including relapsing-remitting, chronic progressive, etc., and the S1P receptor agonists can be used alone or in conjunction with other agents to relieve signs and symptoms of the disease as well as prophylactically.

In addition, the disclosed compounds can be used for altering lymphocyte trafficking as a method for prolonging allograft survival, for example solid organ transplants, treatment of graft vs. host disease, bone marrow transplantation, and the like.

In addition, the disclosed compounds can be used to inhibit autotaxin. Autotaxin, a plasma phosphodiesterase, has been demonstrated to undergo end product inhibition. Autotaxin hydrolyzes several substrates to yield lysophosphatidic acid and sphingosine 1-phosphate, and has been implicated in cancer progression and angiogenesis. Therefore, S1P receptor agonist pro-drugs of the disclosed compounds can be used to inhibit autotaxin. This activity may be combined with agonism at S1P receptors or may be independent of such activity.

In addition, disclosed compounds can be useful for inhibition of S1P lyase. S1P lyase is an intracellular enzyme that irreversibly degrades S1P. Inhibition of S1P lyase disrupts lymphocyte trafficking with concomitant lymphopenia. Accordingly, S1P lyase inhibitors can be useful in modulating immune system function. Therefore, the disclosed compounds can be used to inhibit S1P lyase. This inhibition could be in concert with S1P receptor activity, or be independent of activity at any S1P receptor.

In addition, disclosed compounds can be useful as antagonists of the cannabinoid CB₁ receptor. CB₁ antagonism is associated with a decrease in body weight and an improvement in blood lipid profiles. The CB₁ antagonism could be in concert with S1P receptor activity, or be independent of activity at any S1P receptor.

In addition, disclosed compounds can be useful for inhibition of group IVA cytosolic PLA₂ (cPLA₂). cPLA₂ catalyzes the release of eicosanoic acids (e.g., arachidonic acid). The eicosanoic acids are transformed to pro-inflammatory eicosanoids such as prostaglandins and leukotrienes. Thus, disclosed compounds may be useful as anti-inflammatory agents. This inhibition could be in concert with S1P receptor activity, or be independent of activity at any S1P receptor.

In addition, disclosed compounds may be useful for inhibition of the multiple substrate lipid kinase (MuLK). MuLK is highly expressed in many human tumor cells and thus its inhibition might slow the growth or spread of tumors.

“Treatment” of multiple sclerosis includes the various forms of the disease including relapsing-remitting, chronic progressive, etc., and the S1P receptor agonists can be used alone or in conjunction with other agents to relieve signs and symptoms of the disease as well as prophylactically.

The present invention is also includes pharmaceutical compositions comprising the compounds of formula I. More particularly, such compounds can be formulated as pharmaceutical compositions using standard pharmaceutically acceptable carriers, fillers, solubilizing agents and stabilizers known to those skilled in the art. For example, a pharmaceutical composition comprising a compound of formula I, or analog, derivative, or modification thereof, as described herein, is used to administer the appropriate compound to a subject.

The compounds of formula I are useful for treating a disease or disorder including administering to a subject in need thereof of a therapeutically acceptable amount of a compound of formula I, or a pharmaceutical composition comprising a therapeutically effective amount of a compound of formula I, and a pharmaceutically-acceptable carrier.

The disclosed compounds and method are directed to sphingosine 1-phosphate (S1P) analogs that have activity as receptor agonists or antagonists at one or more S1P receptors, specifically the S1P₁, S1P₄ and S1P₅ receptor types. The disclosed compounds and method include both compounds that have a phosphate moiety as well as compounds with hydrolysis-resistant phosphate surrogates such as phosphonates, alpha-substituted phosphonates particularly where the alpha substitution is a halogen and phosphothionates.

The values listed below for radicals, substituents, and ranges, are for illustration only; they do not exclude other defined values or other values within defined ranges for the radicals and substituents.

X¹, Y¹ and Z¹ are independently O, CH, CH₂, CHCF₃, N, NH, or S.

Another value for X¹, Y¹ and Z¹ is CH₂.

R¹ can be hydrogen, fluorine, chlorine, bromine, trifluoromethyl, methoxy, (C₁-C₆)alkyl, (C₁-C₆)haloalkyl, or (C₁-C₆)alkyl substituted with, alkoxy or cyano.

Additional values for R¹ are hydrogen, trifluoromethyl, or —CH₂CF₃.

More additional values for R¹ are alkyl-substituted aryl, aryl-substituted alkyl, or aryl-substituted arylalkyl.

More additional values for R¹ are benzyl, phenylethyl, or methyl benzyl.

Compounds having formula I can have an R² group that includes a chain having the structure —CH₂—CH₂—O—CH₂—CH₂—O—.

Values for R² include

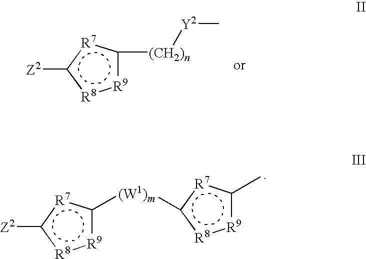

An exemplary value for W′ is a bond, —CH₂—CH₂—CH₂— or —(C═O)(CH₂)₁₋₅.

Additional values for R² having formula II are

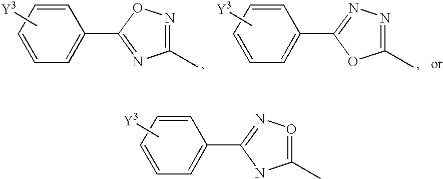

where Y³ is (CH₃)₃C—, CH₃CH₂(CH₃)₂C—, CH₃CH₂CH₂—, CH₃(CH₂)₂CH₂—, CH₃(CH₂)₄—CH₂—, (CH₃)₂CHCH₂—, (CH₃)₃CCH₂—, CH₃CH₂O—, (CH₃)₂CHO—, or CF₃CH₂CH₂— or a group having the formula:

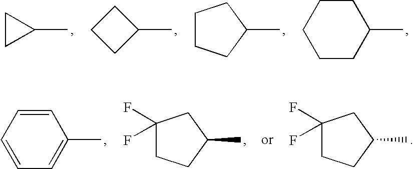

An additional value for R² having formula II (para substituted 3,5-diphenyl-(1,2,4)-oxadiazole) is;

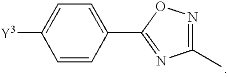

Another value for R² having formula II is;

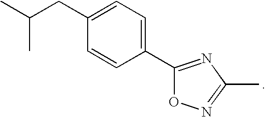

Another value for R² having formula II is;

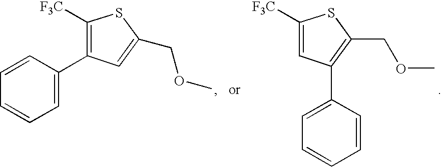

Additional values for R² having formula III are;

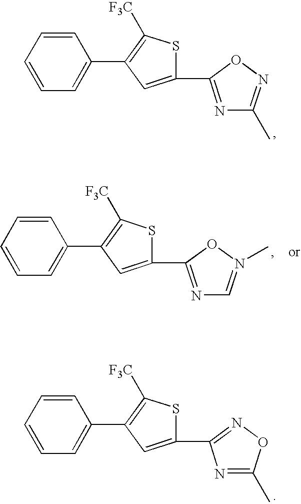

Another value for R² having formula III is;

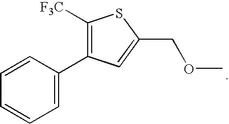

Another value for R² having formula V is;

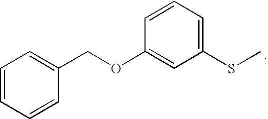

Additional values for R² include (C₁-C₂₀)alkyl, (C₁-C₂₀)alkoxy, or (C₂-C₂₆)alkoxyalkyl.

More additional values for R² include (C₁-C₁₀)alkyl, (C₂-C₁₀)alkenyl and (C₂-C₁4)alkynyl or (C₁-C₁₀)alkoxy optionally substituted with carbonyl (C═O) or oxime (C═NR^{d}) groups.

Additional values for R² include methyl, ethyl, propyl, butyl, pentyl, hexyl, heptyl, octyl, trifluoromethyl, trifluoroethyl, trifluoromethoxy, trifluoroethoxy, methoxy, ethoxy, propoxy, butoxy, pentoxy, heptoxy, or octoxy.

Another value for R³ is methyl, hydroxymethyl, ethyl, hydroxyethyl, propyl, hydroxypropyl, or isopropyl.

Another value for R³ is methyl, hydroxymethyl, ethyl, or hydroxyethyl.

A value for R⁴ is hydroxy, or phosphate (—OPO₃H₂).

A specific compound has the formula

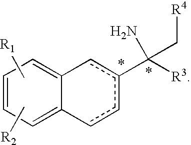

Additional compounds have formulas

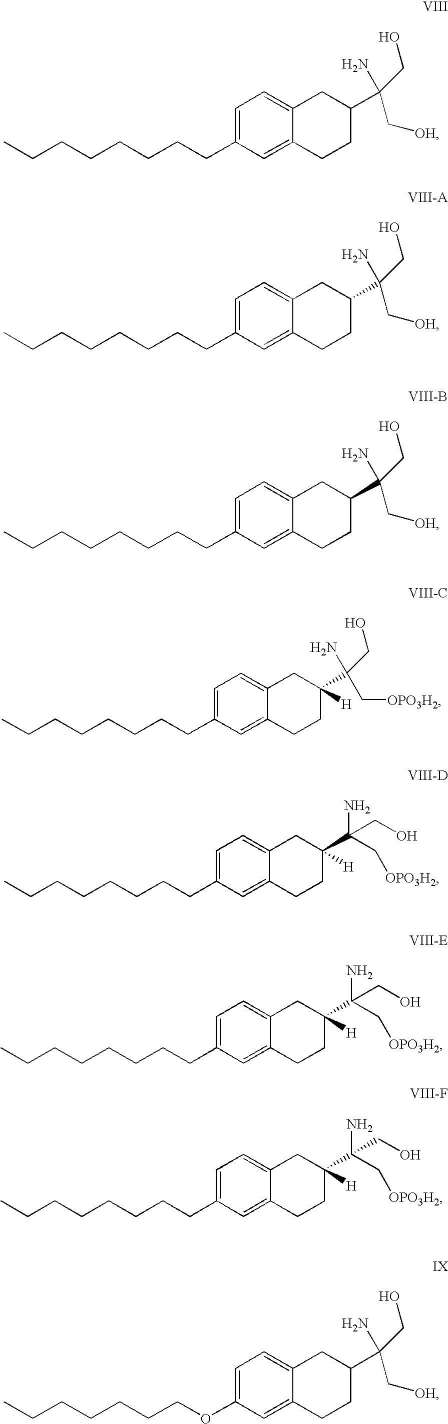

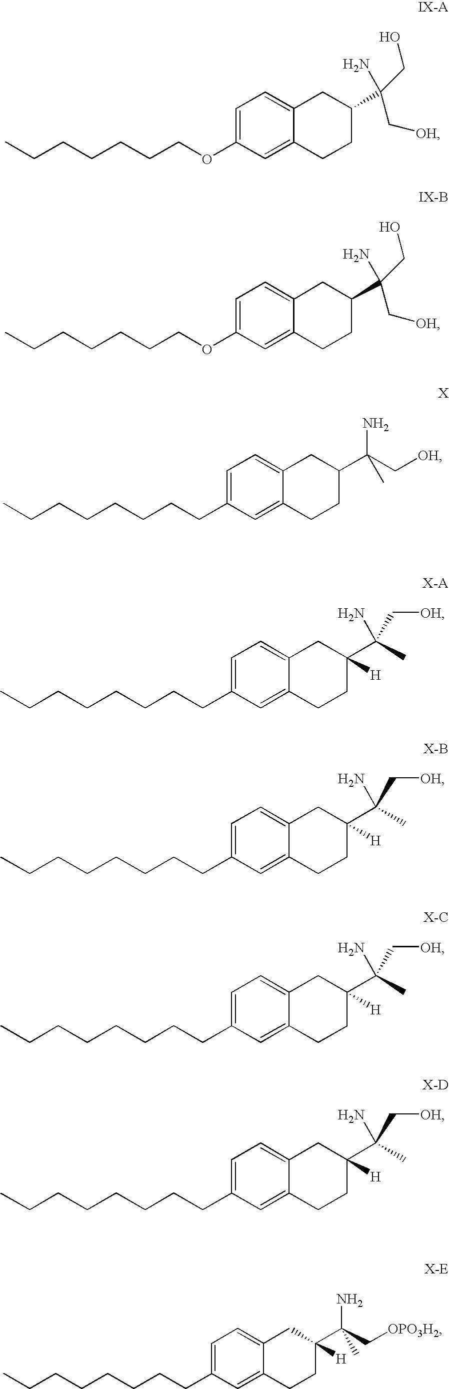

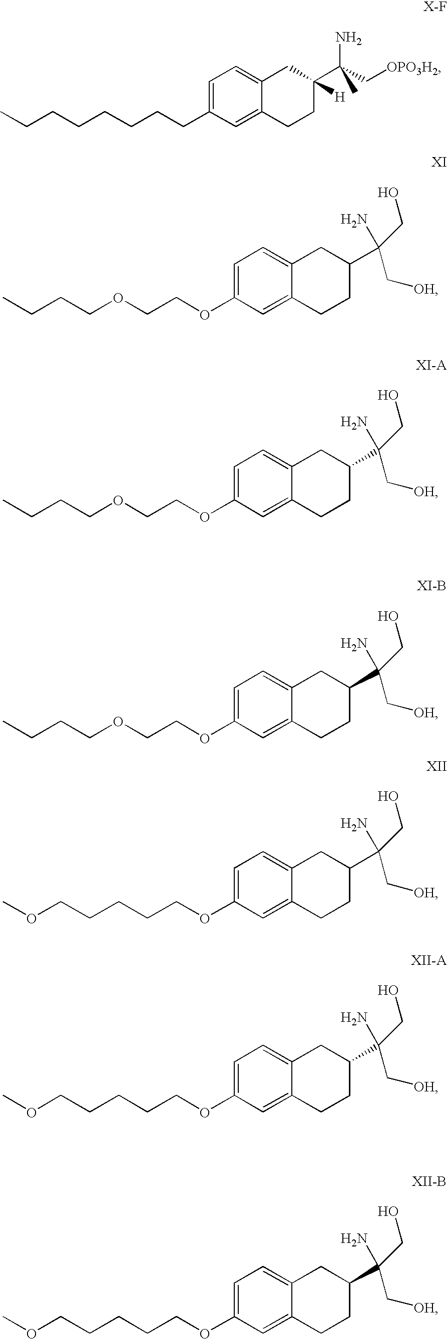

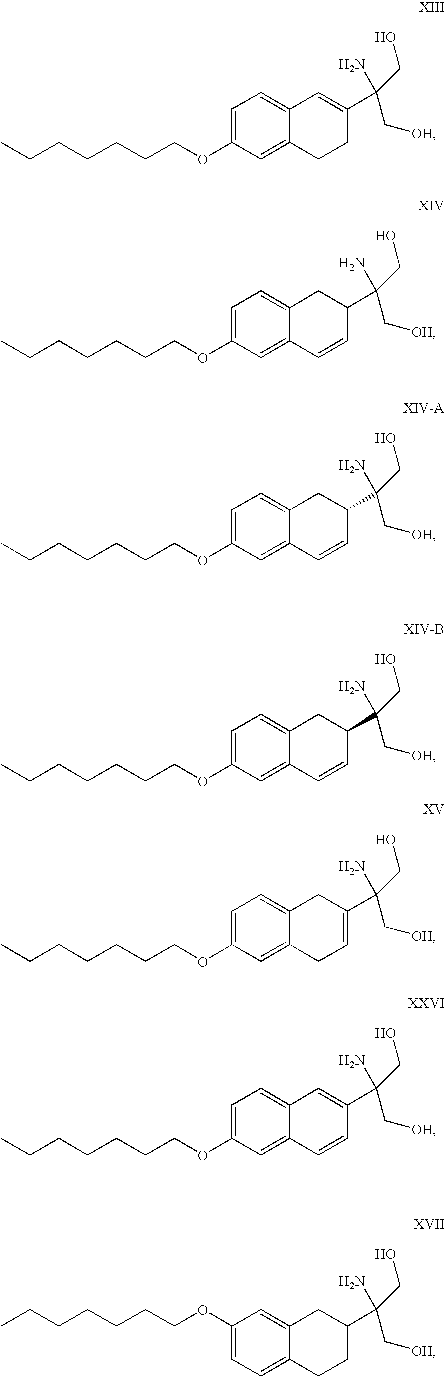

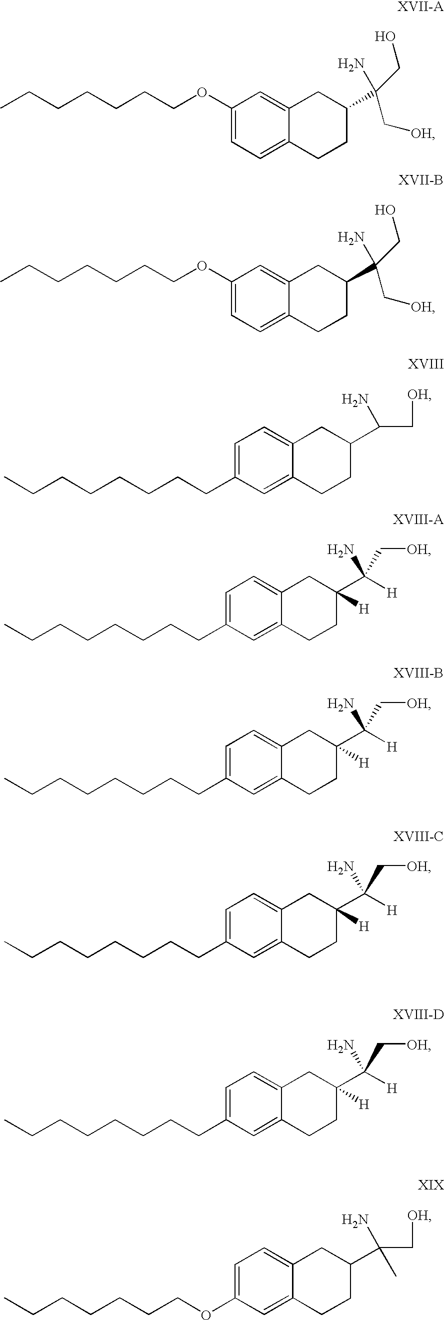

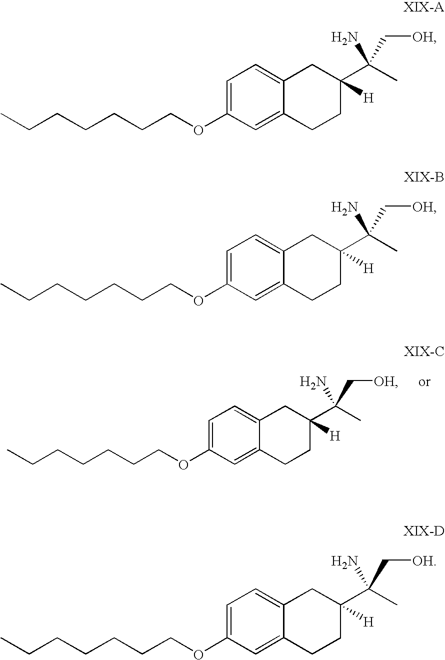

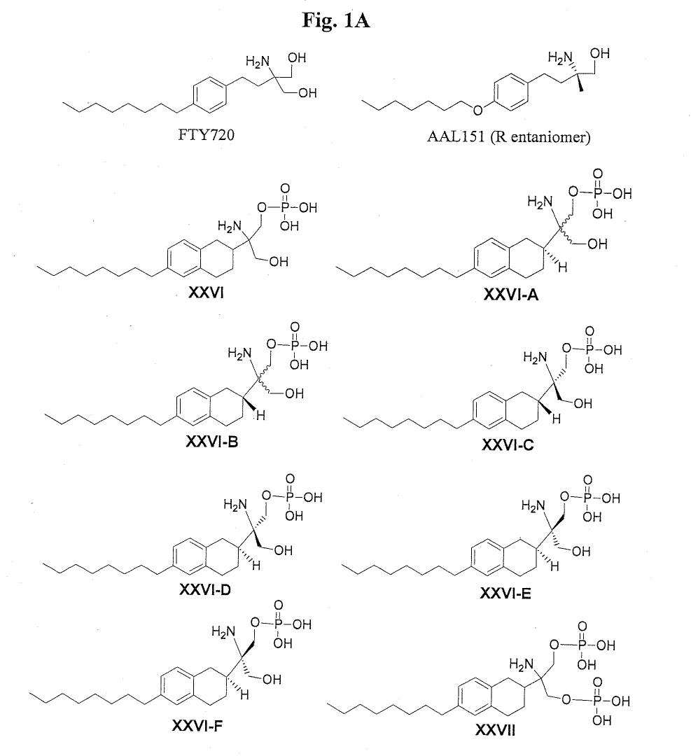

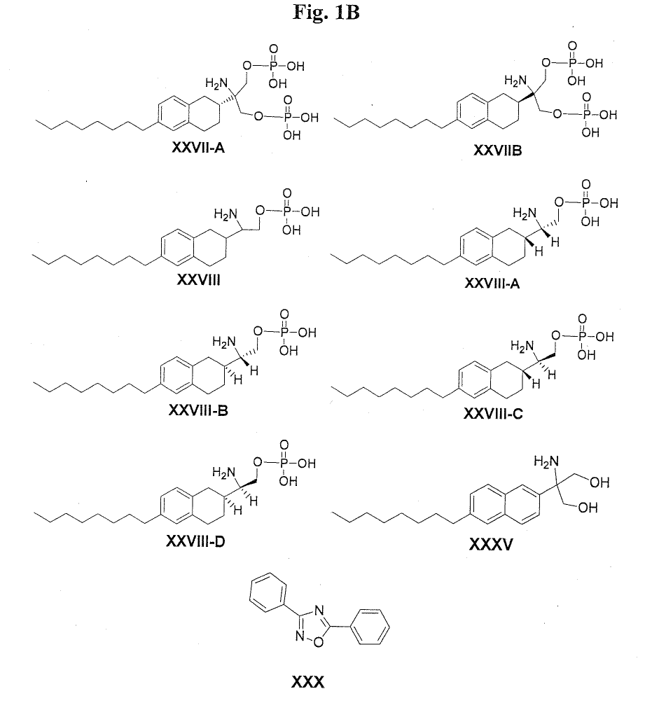

Additional compounds having formula I include compounds above or in FIG. 1 where one of more of the hydrogen atoms from a hydroxy group is replaced with a phosphate group —OP(═O)(OH)₂ and all enantiomers thereof

Additional compounds of formula I are illustrated in table 1, below.

TABLE 1

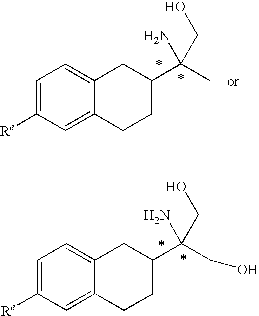

| Compound | R^{e} |
|---|---|
| XX | 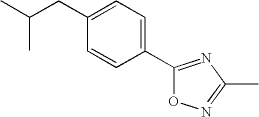 |
| XXI | 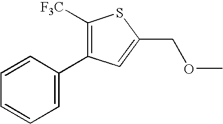 |
| XXII | 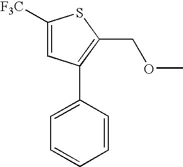 |
| XXIII | 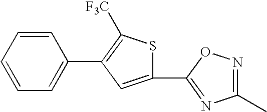 |
| XXIV | 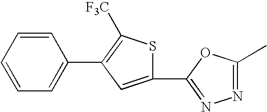 |
| XXV | 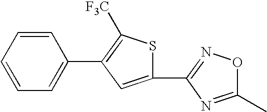 |
| XXXI | 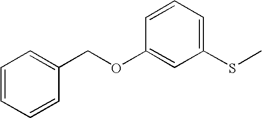 |

The compounds having formulas XX through XXV or XXXI also include all enantiomers thereof such as:

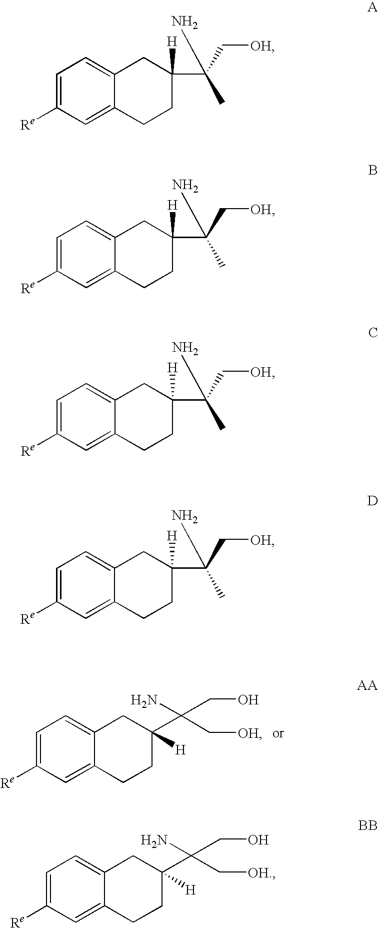

with each of the R^{e} groups from Table 1.

In another aspect, the invention provides S1P receptor pro-drug compounds having the general structure of formula I, is provided by a compound with a mono-substituted tetralin ring system that has the formula VII. In some embodiments of formula I, the structure (e.g., VIII and IX) has only a single chiral center and that the amino carbon is pro-chiral, e.g., will become chiral following enzyme-catalyzed phosphorylation.

Without wishing to be bound by any particular theory, it is expected that the compounds described herein are pro-drugs, e.g., are activated by phosphorylation of the primary alcohol to form the mono-phosphorylated analog. Additionally, the active drugs are expected to be agonists at the S1P type 1 receptor.

In cases where compounds of formula I are sufficiently basic or acidic to form stable nontoxic acid or base salts, preparation and administration of the compounds as pharmaceutically acceptable salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Pharmaceutically-acceptable base addition salts can be prepared from inorganic and organic bases. Salts from inorganic bases, include but are not limited to, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are alkyl, substituted alkyl, alkenyl, substituted alkenyl, cycloalkyl, substituted cycloalkyl, cycloalkenyl, substituted cycloalkenyl, aryl, heteroaryl, or heterocyclic and the like. Also included are amines where the two or three substituents, together with the amino nitrogen, form a heterocyclic or heteroaryl group. Mon-limiting examples of amines include, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful, for example, carboxylic acid amides, including carboxamides, lower alkyl carboxamides, dialkyl carboxamides, and the like.

The compounds of formula I can be formulated as pharmaceutical compositions and administered to a mammalian host, such as a human patient in a variety of forms adapted to the chosen route of administration, e.g., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, e.g., when they are liquids. However, it will generally be desirable to administer them to the skin as compositions or formulations, in combination with a dermatologically acceptable carrier, which may be a solid or a liquid.

Exemplary solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of formula I in a liquid composition, such as a lotion, will be from about 0.1 to about 25 weight percent, preferably from about 0.5-10 weight percent. The concentration in a semi-solid or solid composition such as a gel or a powder will be about 0.1-5 wt-%, preferably about 0.5-2.5 weight percent based on the total weight of the composition.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician. In general, however, a dose will be in the range of from about 0.1 to about 10 mg/kg of body weight per day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four, or more sub-doses per day. The sub-dose itself may be further divided, e.g., into a number of discrete loosely spaced administrations; such as multiple inhalations from an insufflator or by application of a plurality of drops into the eye.

The disclosed method includes a kit comprising an inhibitor compound of formula I and instructional material which describes administering the inhibitor compound or a composition comprising the inhibitor compound to a cell or a subject. This should be construed to include other embodiments of kits that are known to those skilled in the art, such as a kit comprising a (preferably sterile) solvent for dissolving or suspending the inhibitor compound or composition prior to administering the compound or composition to a cell or a subject. Preferably, the subject is a human.

In accordance with the disclosed compounds and methods, as described above or as discussed in the Examples below, there can be employed conventional chemical, cellular, histochemical, biochemical, molecular biology, microbiology, and in vivo techniques which are known to those of skill in the art. Such techniques are explained fully in the literature.

Without further description, it is believed that one of ordinary skill in the art can, using the preceding description and the following illustrative examples, make and utilize the disclosed compounds.

Processes for preparing compounds of formula I or for preparing intermediates useful for preparing compounds of formula I are provided as further embodiments. Intermediates useful for preparing compounds of formula I are also provided as further embodiments. The processes are provided as further embodiments and are illustrated in the schemes herein wherein the meanings of the generic radicals are as given above unless otherwise qualified.

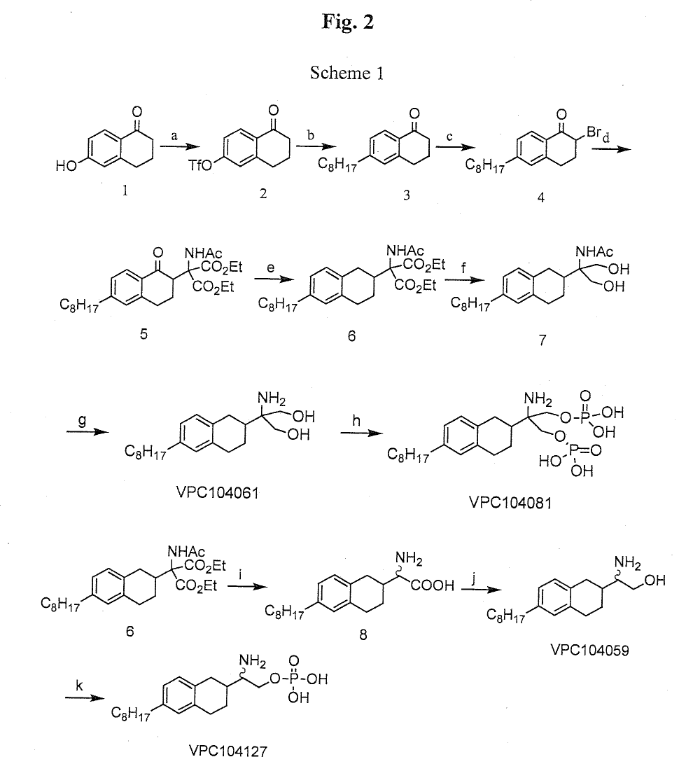

An example of the synthesis of several disclosed compounds is illustrated in Scheme 1 (FIG. 2). The reagents and conditions are as follows: a) Tf₂O, 2,6-lutidine, CH₂Cl₂, 0° C., 2 h, 93%; b) 1-octene, 9-BBN, K₃PO₄, KBr, H₂O, Pd(PPh₃)₄, 65° C., 2 h, 82%; c) CuBr₂, EtOAc, CHCl₃, reflux 6 h, 80%; d) NaH, N-acetamido-diethylmalonate, DMF, 0° C.-rt, overnight, 75%; e) Et₃SH, TiCl₄, CH₂Cl₂, rt, 12 h, 65%; f) LiBH₄, rt, THF, 48 h, 33%; g) LiOH, H₂O, MeOH, THF, 50° C., 5 h, 75%; h) P₂O₅, H₃PO₄, 100° C., 2 h, 37%; i) 12M HCL, MeOH, reflux, 2 h; j) LiAlH₄, THF, reflux, 12 h, 21%, two steps; k) P₂O₅, H₃PO₄, 100° C., 2 h, 50%.

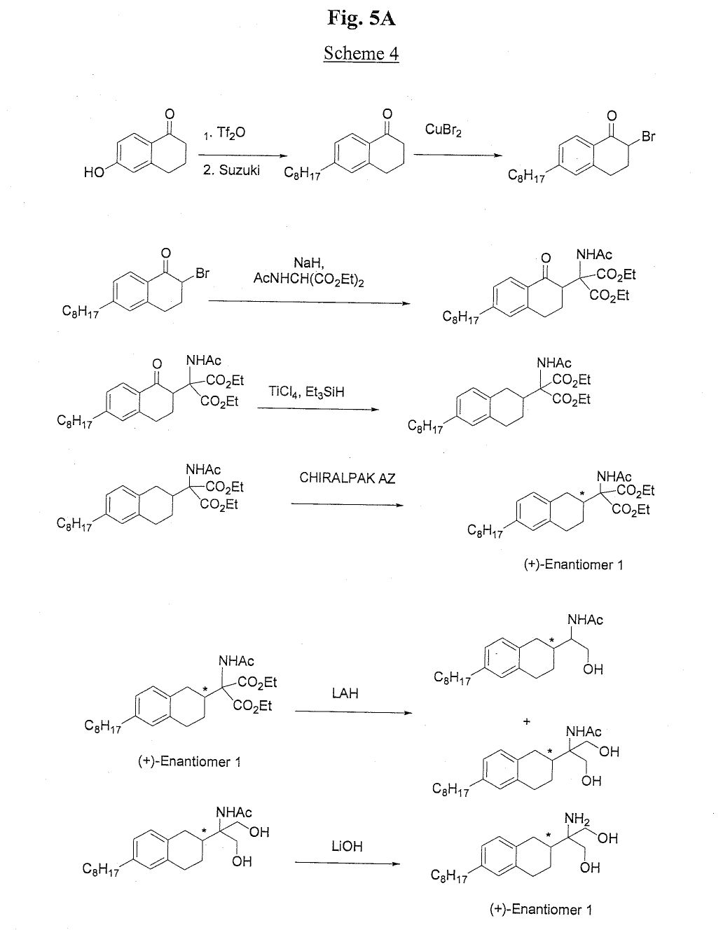

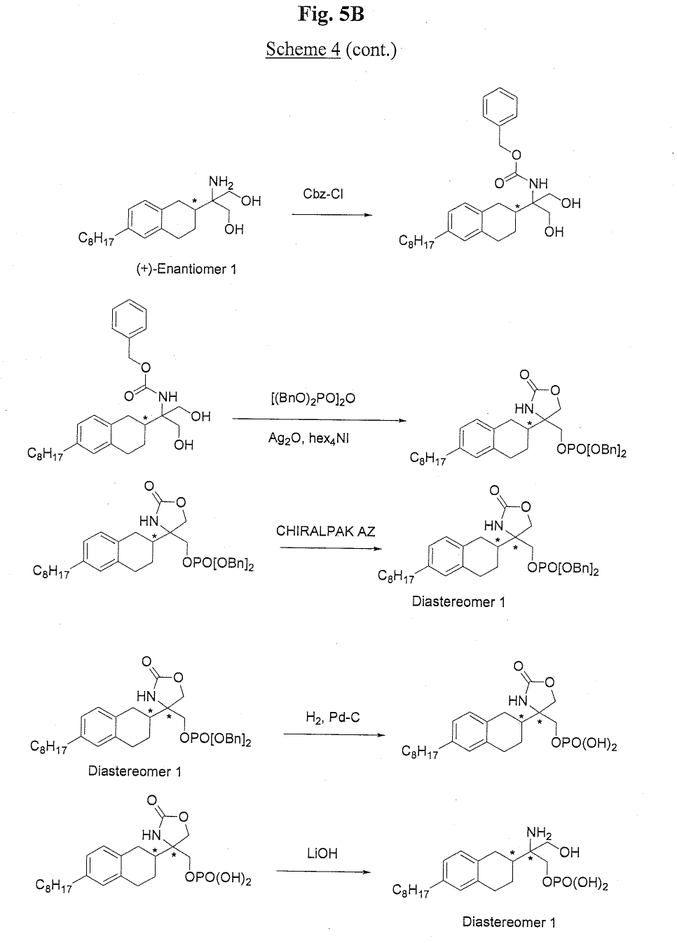

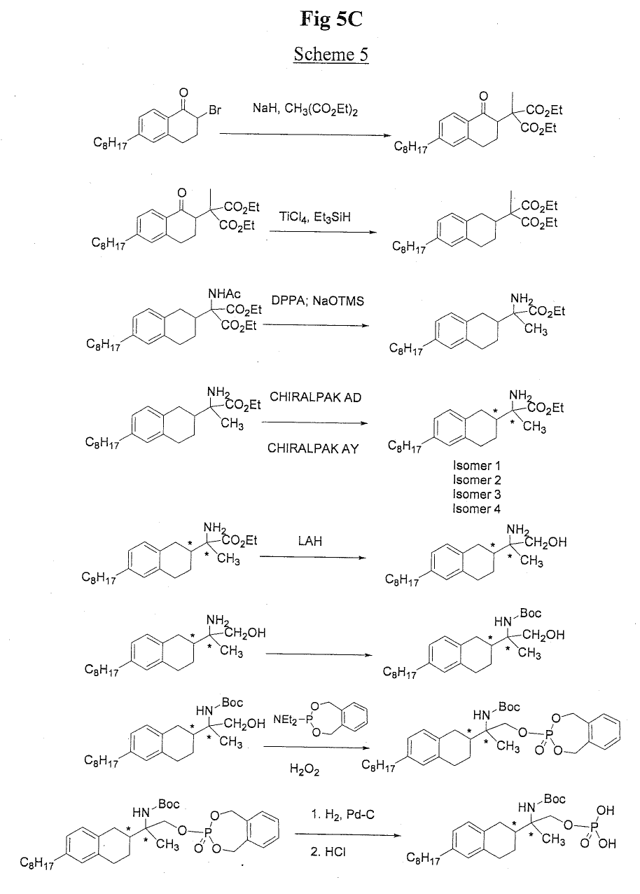

Examples of the syntheses of disclosed compounds that are optically active are illustrated in Schemes 4 and 5 (FIGS. 5A, 5B and 5C) and the examples, below.

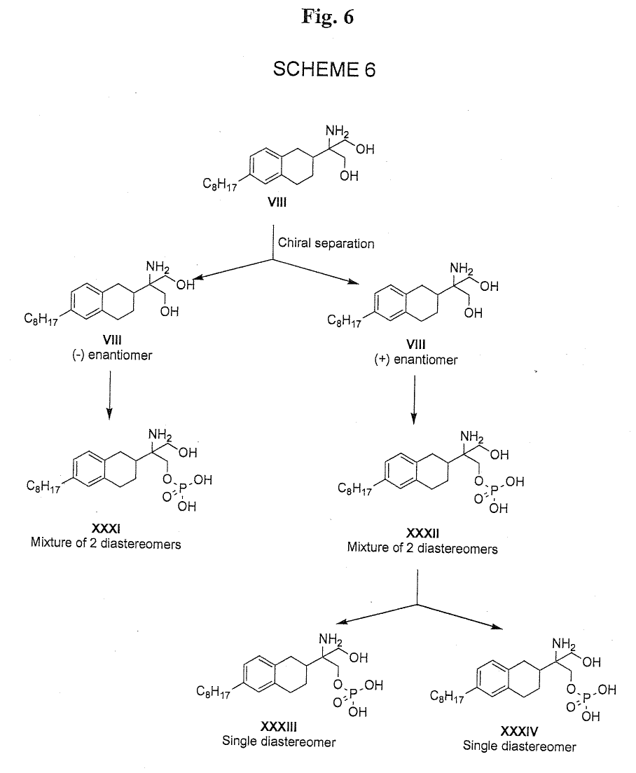

The compounds of the invention were separated into enantiomers using chromatography. Several were also phosphorylated and the phosphorylated compounds also separated into single diastereomers using chiral chromatography. This is illustrated in Scheme 6 (FIG. 6).

The invention is now described with reference to the following Examples and Embodiments. Without further description, it is believed that one of ordinary skill in the art can, using the preceding description and the following illustrative examples, make and utilize the disclosed compounds. The chemicals used are readily available from commercial suppliers, e.g., Sigma-Aldrich. The following working examples therefore, are provided for the purpose of illustration only and specifically point out the preferred embodiments, and are not to be construed as limiting in any way the remainder of the disclosure. Therefore, the examples should be construed to encompass any and all variations which become evident as a result of the teaching provided herein.

### EXAMPLES

### Example 1

### Trifluoromethanesulfonic acid 5-oxo-5,6,7,8-tetrahydro-naphthalen-2-yl ester (2)

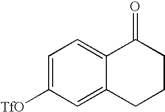

Trifluoromethanesulfonic anhydride (1.7 mL, 10 mmol) was added slowly over 1 hour to a solution of 6-hydroxy-1-tetralone (1.62 g, 10 mmol) and 2,6-lutidine (1.28 mL, 10 mmol) in dry dichloromethane (10 mL) cooled to 0° C. After 1 hour the solution was diluted with dichloromethane (10 mL) and washed with 1 M hydrochloric acid (20 mL). The aqueous layer was re-extracted with dichloromethane (50 mL) and the combined organics washed with 1 M hydrochloric acid (10 mL). The organics were dried over magnesium sulfate and concentrated under vacuum. The residue was purified by column chromatography (Silica gel, CH₂Cl₂) to provide 2.7 g of compound 2 (93%).

¹H NMR (300 MHz, CDCl₃) δ 2.13 (p, 2H, J=6.22 Hz), 2.63 (t, 2H, J=6.95 Hz), 2.98 (t, 2H, J=6.22 Hz), 7.15 (m, 2H), 8.07 (m, 1H); ¹³C NMR δ 23.08, 29.88, 38.92, 116.74, 119.81, 121.56, 130.14, 132.58, 147.38, 152.52, 196.53.

### Example 2

### 6-Octyl-3,4-dihydro-2H-naphthalen-1-one (3)

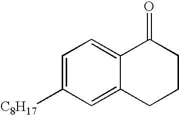

9-BBN (0.5 M solution in THF, 20.2 mL, 10.1 mmol) was added to 1-octene (1.6 mL, 10.1 mmol) at room temperature. The solution was stirred, at room temperature, overnight. After this time, K₃PO₄ (2.93 g, 13.8 mmol), Pd(Ph₃P)₄ (191 mg, 0.17 mmol, 1.8 mol %), KBr (1.2 g, 10.1 mmol) and degassed H₂O (0.18 mL, 10 mmol) were added. This was followed by a solution of compound 2 (2.7 g, 9.2 mmol) in dry degassed THF (10 mL). The reaction mixture was heated at 65° C. under argon for 2 hours. After cooling, the solution was acidified to pH 1 and extracted into EtOAc (100 mL). The aqueous layer was re-extracted with EtOAc (50 mL) and the combined organics washed with H₂O (20 mL) and brine (40 mL). The organic layer was dried over magnesium sulfate and concentrated under vacuum. The residue was purified by column chromatography (Silica gel, 5% EtOAc in hexanes) to provide 1.93 g of compound 3 (82%).

¹H NMR (300 MHz, CDCl₃) δ 0.85 (t, 3H, J=6.95 Hz), 1.24 (bs, 10H), 1.58 (p, 2H, J=6.95 Hz), 2.06 (p, 2H, J=5.85 Hz), 2.57 (t, 4H, J=6.95 Hz), 2.87 (t, 2H, J=6.22 Hz), 7.01 (s, 1H), 7.06 (d, 1H, J=8.05 Hz), 7.91 (d, 1H, J=8.06 Hz); ¹³C NMR δ 14.32, 22.88, 23.61, 29.44, 29.55, 29.66, 29.96, 31.32, 32.08, 36.31, 39.33, 127.12, 127.45, 128.73, 130.75, 144.70, 149.28, 198.09.

### Example 3

### 2-Bromo-6-octyl-3,4-dihydro-2H-naphthalen-1-one (4)

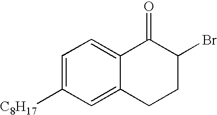

Cupric bromide (3.34 g, 15.0 mmol) was heated at reflux in ethyl acetate (10 mL) with stirring. To this was added compound 3 (1.93 g, 7.5 mmol) in chloroform (10 mL). The reaction was heated at reflux for a further 6 hours and cooled. Copper bromide and cupric bromide residues were filtered off and the filtrate was decolorized with activated charcoal and filtered through a bed of Celite and washed with ethyl acetate (4×50 mL). The solvent was removed under reduced pressure and the residue was purified by column chromatography (Silica gel, 2% EtOAc in hexanes) to provide 2.02 g of compound 4 (80%).

¹H NMR (300 MHz, CDCl₃) δ 0.87 (t, 3H, J=6.95 Hz), 1.26 (bs, 10H), 1.61 (p, 2H, J=6.96 Hz), 2.46 (m, 2H), 2.62 (t, 2H, J=7.69 Hz), 2.86 (dt, 1H, J=16.34 Hz, 4.39 Hz), 3.27 (dt, 1H, J=16.83 Hz, 4.39 Hz), 4.69 (t, 1H, J=4.02 Hz), 7.07 (s, 1H), 7.14 (d, 1H, J=8.05 Hz), 7.99 (d, 1H, J=8.05 Hz); ¹³C NMR δ 14.34, 22.88, 26.42, 29.44, 29.57, 29.64, 31.25, 32.08, 32.32, 36.39, 127.75, 128.00, 128.73, 129.00, 144.30, 150.39, 190.54.

### Example 4

### 2-Acetylamino-2-(6-octyl-1-oxo-1,2,3,4-tetrahydro-naphthalen-2-yl)-malonic acid diethyl ester (5)

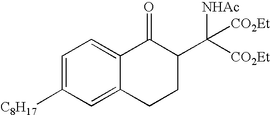

Sodium hydride (720 mg, 18.0 mmol) 60% in mineral oil was suspended in dry DMF (10 mL) and a solution of diethyl acetamidomalonate (3.26 g, 15 mmol) in dry DMF (10 mL) was added. The solution was stirred at 0° C. for 3 hours until the anion had formed. A solution of 4 (2.02 g, 6.0 mmol) in dry DMF (10 mL) was added and the solution warmed to room temperature and stirred overnight. The mixture was poured into distilled water (50 mL), in an ice-bath, acidified to pH 3 with 1M hydrochloric acid and extracted with ethyl acetate (3×50 mL). The organic phases were washed with brine (2×30 mL) and dried over magnesium sulfate and concentrated under vacuum. The residue was purified by column chromatography (Silica gel, 40% EtOAc in hexanes) to provide 2.12 g of compound 5 (75%).

¹H NMR (300 MHz, CDCl₃) δ 0.85 (t, 3H, J=6.22 Hz), 1.24 (m, 16H), 1.58 (p, 2H, J=6.95 Hz), 1.97 (s, 3H), 2.59 (t, 2H, J=7.32 Hz), 2.83-3.21 (m, 4H), 3.88 (dd, 1H, J=14.00 Hz, 3.68 Hz), 4.14-4.32 (m, 4H), 6.86 (s, 1H), 7.03 (s, 1H), 7.07 (d, 1H, J=8.69 Hz), 7.84 (d, 1H, J=8.36 Hz); ¹³C NMR δ 14.05, 14.16, 14.30, 22.85, 23.31, 26.98, 29.40, 29.49, 29.61, 29.98, 31.28, 32.05, 36.32, 56.16, 62.40, 63.13, 66.33, 127.16, 127.63, 128.78, 144.84, 150.07, 166.38, 168.70, 169.83, 197.63.

### Example 5

### 2-Acetylamino-2-(6-octyl-1,2,3,4-tetrahydro-naphthalen-2-yl)-malonic acid diethyl ester (6)

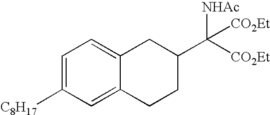

To a solution of triethylsilane (1.3 ml, 8.2 mmol) in 5 ml of CH₂Cl₂ was added compound 5 (1 g, 2.1 mmol) in 5 ml of CH₂Cl₂. The reaction mixture was stirred at mom temperature under Ar and TiCl₄ (0.09 ml, 8.2 mmol) was added dropwise. The resulting solution was stirred for 12 hours, cooled to 0° C. and quenched by slow addition of 10 ml of saturated NaHCO₃. The aqueous layer was extracted with CH₂Cl₂ (3×30 mL). The combined organic layers were washed with brine (2×30 mL) and dried over magnesium sulfate and concentrated under vacuum. The residue was purified by column chromatography (Silica gel, 20% EtOAc in hexanes) to provide 630 mg of compound 6 (65%).

¹H NMR (300 MHz, CDCl₃) δ 0.87 (t, 3H, J=6.46 Hz), 1.26 (m, 16H), 1.58 (p, 2H, J=6.79 Hz), 2.03 (s, 3H), 2.28 (b, 1H), 2.49-2.68 (m, 4H), 2.82-2.92 (m, 2H), 4.20-4.34 (m, 4H), 6.69 (s, 1H), 6.89-7.05 (m, 3H); ¹³C NMR δ 14.21, 14.25, 14.37, 22.92, 23.37, 25.48, 29.50, 29.63, 29.72, 29.76, 30.39, 31.93, 32.13, 35.78, 40.33, 62.46, 62.79, 68.80, 126.04, 128.81, 129.30, 132.85, 136.28, 140.66, 150.07, 167.63, 168.32, 169.42.

### Example 6

### N-[2-Hydroxy-1-hydroxymethyl-1-(6-octyl-1,2,3,4-tetrahydro-naphthalen-2-yl)-ethyl]acetamide (7)

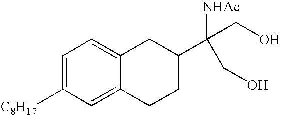

Lithium borohydride (2M solution in THF, 0.88 ml, 1.76 mmol) was added to compound 6 (200 mg, 0.44 mmol) in 5 ml THF at 0° C. The reaction mixture was stirred at room temperature for 48 hours and diluted with 40 ml ethyl acetate. The solution was washed with brine (2×20 mL) and dried over magnesium sulfate and concentrated under vacuum. The residue was purified by column chromatography (Silica gel, 4% MeOH in CH₂Cl₂) to provide 55 of mg compound 7 (33%).

¹H NMR (300 MHz, CDCl₃) δ 0.88 (t, 3H, J=6.56 Hz), 1.29 (m, 10H), 1.57 (p, 2H, J=6.25 Hz), 1.94-1.98 (m, 2H), 2.05 (s, 3H), 2.33 (m, 1H), 2.51 (t, 2H, J=7.32), 2.60-2.85 (m, 4H), 3.69 (d, 2H, J=11.61), 3.89 (dd, 2H, J=11.61 Hz, 7.25 Hz), 6.22 (s, 1H), 6.88-6.99 (m, 3H); ¹³C NMR δ 14.38, 22.92, 24.20, 24.35, 29.52, 29.66, 29.73, 29.95, 30.32, 31.94, 32.14, 35.78, 38.26, 63.55, 64.34, 64.46, 126.18, 128.85, 129.30, 133.06, 136.22, 140.75, 172.40.

### Example 7

### 2-Amino-2-(6-octyl-1,2,3,4-tetrahydro-naphthalen-2-yl)-propane-1,3-diol (VPC104061)

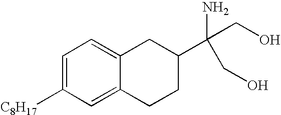

A solution of compound 7 (53 mg, 0.14 mmol) and LiOH. H₂O (45 mg, 1.1 mmol) in MeOH (3 ml), THF (1.5 ml) and water (3 ml) was stirred at 50° C. for 5 hours and diluted with ethyl acetate (20 ml). The solution was washed with brine (2×10 mL) and dried over magnesium sulfate and concentrated under vacuum. The residue was purified by column chromatography (Silica gel, 50% MeOH in CH₂Cl₂) to provide 35 mg of compound VPC104061 (75%).

¹H NMR (300 MHz, CDCl₃) δ 0.88 (t, 3H, J=6.17 Hz), 1.29 (m, 10H), 1.56 (p, 2H, J=6.17 Hz), 1.82-1.98 (m, 2H), 2.51 (t, 2H, J=6.95), 2.58-2.88 (m, 5H), 3.19 (b, 4H), 3.61 (d, 2H, J=10.98), 3.73 (d, 2H, J=10.61 Hz), 6.87-6.98 (m, 3H); ¹³C NMR δ 14.37, 22.93, 24.02, 29.32, 29.53, 29.70, 29.75, 29.85, 30.26, 31.94, 32.14, 35.08, 39.58, 57.74, 66.13, 66.19, 126.09, 128.81, 129.39, 133.28, 136.26, 140.64. MS (ESI) m/z 334.1 [M+H]⁺.

### Example 8

### Phosphoric acid mono-[2-amino-2-(6-octyl-1,2,3,4-tetrahydro-naphthalen-2-yl)-3-phosphonooxypropyl]ester (VPC104081)

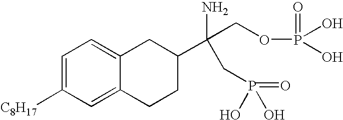

Phosphorus pentoxide (2.0 g, 14 mmol) in phosphoric acid (85% in water, 2 ml, 29 mmol) was added to VPC104061 (25 mg, 0.07 mmol). The mixture was stirred at 100° C. for 2 hours and cooled to 0° C. The product was precipitated by adding water (14 mg, 37%). MS (ESI) m/z 494.4 [M+H]⁺.

### Example 9

### Amino-(6-octyl-1,2,3,4-tetrahydro-naphthalen-2-yl)-acetic acid (8)

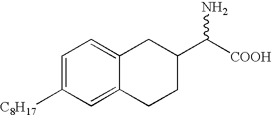

Compound 6 (300 mg, 0.65 mmol) was added to 12 M HCl (10 ml). The mixture was heated to reflux and MeOH (5 ml) was added until the mixture became homogenous. Reflux was continued for 2 hours until the starting material was consumed as determined by thin layer chromatography (TLC). The reaction mixture was concentrated under reduced pressure and co-evaporated with MeOH and diethyl ether multiple times. The desired compound 8 was recrystallized from diethyl ether and hexanes to provide a light brown solid and used directly for the next reaction.

### Example 10

### 2-Amino-2-(6-octyl-1,2,3,4-tetrahydro-naphthalen-2-yl)-ethanol (VPC104059)

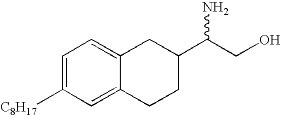

The amino acid 8 prepared in Example 9 was added to a refluxing solution of lithium aluminum hydride (62 mg, 1.63 mmol) in THF (10 ml). The reaction mixture was heated at reflux for 12 hours, subsequently cooled to 0° C. and 10 M NaOH was added and stirred for 20 minutes. Ethyl acetate (20 ml) was added and the mixture was filtered through Celite and magnesium sulfate. The filtrate was concentrated under vacuum and purified by column chromatography (Silica gel, 50% MeOH in CH₂Cl₂) to provide 41 mg of the product, VPC104059 (21%, two steps).

¹H NMR (300 MHz, CDCl₃) δ 0.88 (t, 3H, J=6.39 Hz), 1.28 (m, 10H), 1.55 (p, 2H, J=7.16 Hz), 1.67-2.11 (m, 3H), 2.48 (t, 2H, J=7.69), 2.56-2.83 (m, 5H), 3.19 (b, 4H), 3.47-3.75 (m, 2H), 6.82-6.96 (m, 3H); ¹³C NMR δ 13.34, 22.58, 26.45, 29.14, 29.21, 29.29, 29.47, 31.70 31.90, 32.26, 35.40, 47.56, 125.64, 128.36, 128.86, 128.93, 133.19, 139.93, MS (ESI) m/z 303.9 [M+H]⁺.

### Example 11

### Phosphoric acid mono-[2-amino-2-(6-octyl-1,2,3,4-tetrahydro-naphthalen-2-yl)-ethyl]ester (VPC104127)

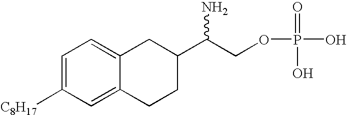

Phosphorus pentoxide (1.5 g, 10.5 mmol) in phosphoric acid (85% in water, 1.5 ml, 22 mmol) was added to VPC104059 (25 mg, 0.08 mmol). The mixture was stirred at 100° C. for 2 h and cooled to 0° C. The product was precipitated by adding water (10 mg, 50%). MS (ESI) m/z 384.2 [M+H]⁺.

### Example 12

### Synthesis of Structure (X)

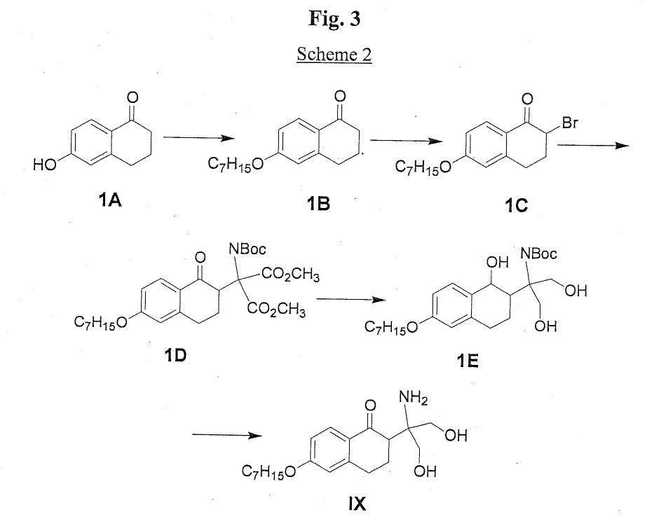

The synthesis of an ether containing compound having formula IX is illustrated in Scheme 2 (FIG. 3). Keto-alcohol 1A, is converted to the keto-ether 1B, using standard reagents and techniques. The keto-ether is halogenated to provide halo-ether 1C in a manner similar to Example 3. The halo-ether is alkylated to provide diester-ether 1D, in a manner similar to the procedure described in Example 4. The diester is converted to ether-triol 1E, using standard reducing agents known in the art. The triol is converted to a diol and deprotected using standard methods known in the art, to provide compound IX.

### Example 13

### 2-Acetylamino-2-(6-octyl-1-hydroxy-1,2,3,4-tetrahydro-naphthalen-2-yl)-malonic acid diethyl ester (A)

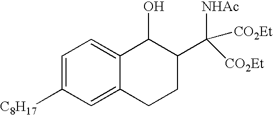

To a solution of sodium borohydride (75 mg, 2.00 mmol) in 5 ml of ethanol at room temperature is added 2-acetylamino-2-(6-octyl-1-oxo-1,2,3,4-tetrahydro-naphthalen-2-yl)-malonic acid diethyl ester (compound 5) (1.00 g, 2.1 mmol) in 5 ml of ethanol. The reaction mixture is stirred at room temperature under argon for an additional hour, quenched by the addition of water (20 mL) and methylene chloride (20 mL). The organic layer is removed and the aqueous layer is extracted with methylene chloride (2×20 mL). The combined organic layers were washed with brine (2×20 mL) and dried over magnesium sulfate and concentrated under vacuum. The residue is purified by column chromatography (silica gel, 20% ethyl acetate in hexanes) to provide 755 mg of compound A (75%).

### Example 14

### 2-Acetylamino-2-(6-octyl-3,4-dihydro-naphthalen-2-yl)-malonic acid diethyl ester (B)

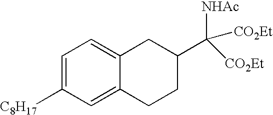

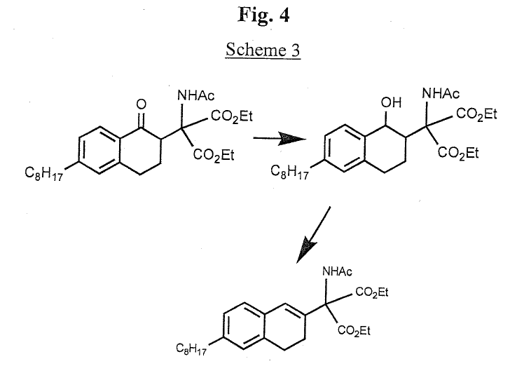

2-Acetylamino-2-(6-octyl-1-hydroxy-1,2,3,4-tetrahydro-naphthalen-2-yl)-malonic acid diethyl ester (compound A, 755 mg, 1.58 mmol)) is dissolved in acetic anhydride (5 mL) followed at 0° C. by a catalytic amount of ferric chloride (66 mg, 0.4 mmol). The reaction is stirred at 0° C. for an additional 2 hours, and 20 mL of diethyl ether is added. The reaction is carefully poured into 50 mL of ice cold water and the organic layer quickly separated. The organic layer is re-extracted with methylene chloride (2×20 mL) and the combined organic layers were washed once with brine (20 mL) and dried over magnesium sulfate. The organic layer is concentrated under vacuum and the residue is purified by column chromatography (silica gel, 10% ethyl acetate in hexanes) to provide 458 mg of compound B (60%). The complete synthesis is illustrated in Scheme 3 (FIG. 4).

### Example 15

### Trifluoro-methanesulfonic acid 5-oxo-5,6,7,8-tetrahydro-naphthalen-2-yl ester

Trifluoromethanesulfonic anhydride (28 mL, 0.17 mol, Aldrich) was slowly added to a solution containing 6-hydroxy-3,4-dihydro-2H-naphthalen-1-one (25.45 g, 0.1569 mol, Aldrich) and 2,6-Lutidine (19 mL, 0.16 mol, Aldrich) in Methylene chloride (200 mL, Acros) at 0° C. Additional 2,6-Lutidine (2 mL, 0.02 mol, Aldrich) was then added and the reaction was stirred at room temperature for three days. The reaction was diluted with methylene chloride and washed once with 1N HCl. The organic layer was then dried with magnesium sulfate and concentrated. The crude material was used without further purification in Example 16. MS: m/z=295.24 M+H.

### Example 16

### 6-Octyl-3,4-dihydro-2H-naphthalen-1-one

9-Borabicyclo[3.3.1]nonane (168 mmol, 0.168 mol, Aldrich) was dissolved in tetrahydrofuran (350 mL, Acros) and 1-octene (28 mL, 0.18 mol, Aldrich) was added. The reaction was stirred at room temperature for 2.5 hours. Potassium bromide (20.5 g, 0.172 mol, Aldrich), tetrakis(triphenylphosphine)palladium(0) (9.1 g, 0.0078 mol, Strem), potassium phosphate (50.0 g, 0.235 mol, Aldrich), Water (3.1 mL, 0.17 mol, Fisher), and trifluoro-methanesulfonic acid 5-oxo-5,6,7,8-tetrahydro-naphthalen-2-yl ester (46.17 g, 0.1569 mol) in 100 mL THF was added. The reaction was covered with a bed of argon and heated at 65° C. for 3 hours. After the reaction was complete the mixture was diluted with ethyl acetate, washed with saturated bicarbonate, washed with 5% citric acid, washed with brine, dried with magnesium sulfate and concentrated. The residue was purified by silica gel chromatography using 0-5% ethyl acetate/hexanes as eluent to provide the title compound in 22.75 g (56% over 2 steps) yield. MS: m/z=259.41 M+H.

### Example 17

### (±)-2-Bromo-6-octyl-3,4-dihydro-2H-naphthalen-1-one

Copper(II) bromide (20.6 g, 0.0924 mol, Aldrich), 6-octyl-3,4-dihydro-2H-naphthalen-1-one (12.00 g, 0.04644 mol), ethyl acetate (60 mL, Fisher) and chloroform (60 mL, Aldrich) were added to a round-bottom flask. The reaction was stirred and heated at reflux overnight. The solution was filtered, decolorized with activated charcoal, filtered through Celite, washed with ethyl acetate and concentrated. The residue was purified by silica gel chromatography using 2-5% ethyl acetate in hexanes as eluent to provide 11.69 g of the title compound (75%) as an oil. MS: m/z=337.33 M+H.

### Example 18

### (±)-2-Acetylamino-2-(6-octyl-1-oxo-1,2,3,4-tetrahydro-naphthalen-2-yl)-malonic acid diethyl ester

Sodium hydride (60% in mineral oil, 4.16 g, 0.104 mol, Aldrich) and N,N-Dimethylformamide (61 mL, Acros) were slurried in a round-bottom flask. The mixture was cooled at 0° C. and 2-acetylamino-malonic acid diethyl ester (18.80 g, 0.08654 mol, Acros) in N,N-dimethylformamide (61 mL, Acros) was added slowly. The reaction was stirred at 0° C. for 3 hours at which point a solution of (±)-2-bromo-6-octyl-3,4-dihydro-2H-naphthalen-1-one (11.69 g, 0.03466 mol) in N,N-dimethylformamide (71 mL, Acros) was added. The reaction was stirred at room temperature for 16 hours. The mixture was diluted with distilled water, cooled in an ice bath and acidified to pH 3 with 1M HCl. The reaction was extracted with ethyl acetate, washed with saturated sodium chloride, dried with magnesium sulfate, filtered and concentrated. The residue was purified by silica gel chromatography using 0-50% ethyl acetate in hexanes as eluent to provide the title compound in 12.88 g (78%) yield. MS: m/z=474.77 M+H.

### Example 19

### (±)-2-Acetylamino-2-(6-octyl-1,2,3,4-tetrahydro-naphthalen-2-yl)-malonic acid diethyl ester

Triethylsilane (17 mL, 0.11 mol, Aldrich) in Methylene chloride (65 mL, Acros) was dissolved in a round-bottom flask. 2-Acetylamino-2-(6-octyl-1-oxo-1,2,3,4-tetrahydro-naphthalen-2-yl)-malonic acid diethyl ester (12.88 g, 0.02720 mol) in methylene chloride (65 mL, Acros) was added to the flask by addition funnel. The mixture was stirred at 0° C. under an argon atmosphere and titanium tetrachloride (12 mL, 0.11 mol, Aldrich) was added slowly by addition funnel. The reaction was stirred and was allowed to warm to room temperature, then was stirred overnight at room temperature. The reaction was cooled at 0° C. and quenched with saturated sodium bicarbonate by slow addition. The mixture was extracted with methylene chloride, washed with saturated sodium chloride, dried over magnesium sulfate, filtered and concentrated. The residue was purified by silica gel chromatography using 0-10% methanol/dichloromethane as eluent to provide 0.994 g (8%) of the title compound. MS: m/z=460.85 M+H.

Impure fractions (9.88 g, 1:1.1 ratio of product to starting material by ¹H NMR) were concentrated, azeotroped with toluene and resubjected to the triethylsilane/titanium chloride conditions, this time adding ¼ of the titanium chloride at room temperature and then the remainder at 0° C. Isolation/purification as before provided 9.07 g (73%) of the title compound, as a 20:1 mixture of product:starting material by ¹H NMR. MS: m/z=460.51 M+H.

### Example 20

### (+)-2-Acetylamino-2-(6-octyl-1,2,3,4-tetrahydro-naphthalen-2-yl)-malonic acid diethyl ester and (−)-2-Acetylamino-2-(6-octyl-1,2,3,4-tetrahydro-naphthalen-2-yl)-malonic acid diethyl ester

Racemic (±)-2-Acetylamino-2-(6-octyl-1,2,3,4-tetrahydro-naphthalen-2-yl)-malonic acid diethyl ester (8.56 g, 18.6 mmol) was separated using CHIRALPAK AZ column, with hexane/isopropanol (84:16) as eluent. Two enantiomers were isolated and carried forward: (+)-ENANTIOMER 1 (Peak 1; 4.11 g, 48% yield; 99.0% ee) was characterized by analytical HPLC(CHIRALPAK AD-H column, 4.6 mm ID×250 mm 85:15 hexane/IPA 1 mL/min gives RT=4.758 min @ 205 nm); MS: m/z=460.62 M+H; specific rotation +13.5 deg (0.5, ethanol). (−)-ENANTIOMER 2 (Peak 2; 3.98 g, 46% yield; 99.6% ee) was characterized by analytical HPLC(CHIRALPAK AD-H column, 4.6 mm ID×250 mm 85:15 hexane/IPA 1 mL/min gives RT=6.088 min @ 205 nm); MS: m/z=460.57 M+H; specific rotation −14.7 deg (0.5, ethanol).

### Example 21

### N-[2-Hydroxy-1-hydroxymethyl-1-(6-octyl-1,2,3,4-tetrahydro-naphthalen-2-yl)-ethyl]acetamide (ENANTIOMER 1)

A 1.00 M solution of Lithium tetrahydroaluminate in Tetrahydrofuran (42.2 mL, Aldrich) was added to Tetrahydrofuran (75 mL, Aldrich) and maintained at 0° C. (+)-2-Acetylamino-2-(6-octyl-1,2,3,4-tetrahydro-naphthalen-2-yl)-malonic acid diethyl ester (ENANTIOMER 1) (6.47 g, 14.1 mmol) in Tetrahydrofuran (50 mL, Aldrich) was added dropwise to the solution. The reaction mixture was stirred at room temperature for 2 hours. The mixture was cooled in an ice/water bath and quenched by slow addition of 1N HCl. The reaction mixture was extracted with ethyl acetate, washed with saturated sodium chloride, dried with sodium sulfate, filtered and evaporated to provide the crude product. The residue was purified by silica gel chromatography using 0-10% methanol in dichloromethane as eluent (Rf=0.16 in 5% methanol/methylene chloride, PMA visualization). The product was isolated in 3.133 g (59%) yield as a 6:1 mixture of product and by-product N-[2-Hydroxy-1-(6-octyl-1,2,3,4-tetrahydronaphthalen-2-yl)-ethyl]-acetamide by ¹H NMR. MS: m/z=376.48 M+H (product), 346.44 M+H (by-product).

### Example 22

### (+)-2-Amino-2-(6-octyl-1,2,3,4-tetrahydro-naphthalen-2-yl)-propane-1,3-diol (VIII-A)

N-[2-Hydroxy-1-hydroxymethyl-1-(6-octyl-1,2,3,4-tetrahydro-naphthalen-2-yl)-ethyl]-acetamide, (ENANTIOMER 1) (4 mg, 0.00111 mol), Lithium hydroxide (221 mg, 0.00923 mol, Fisher) in Methanol (24 mL, Fisher), THF (12 mL, Acros) and Water (24 mL, Fisher) were dissolved in a vial. The starting material was 416 mg total of a 4:1 mixture of starting material and the by-product N-[2-Hydroxy-1-(6-octyl-1,2,3,4-tetrahydro-naphthalen-2-yl)-ethyl]acetamide. The reaction was stirred at reflux for 5 hours. The reaction was cooled, diluted with ethyl acetate, washed with saturated sodium chloride, dried with sodium sulfate, filtered and evaporated. The material was taken up in DMF and purified by preparative HPLC. Appropriate fractions were combined and evaporated to provide the title compound in 250 mg yield (59%) as the HCO₂H salt. MS: m/z=334.52 M+H; specific rotation +58.0 deg (0.1, ethanol); ¹H NMR (500 MHz, DMSO-d6) δ ppm 8.322 (s, 1H) 6.918 (d, J=8.1 Hz, 1H) 6.859 (d, J=8.1 Hz, 1H) 6.840 (s, 1H) 3.537-3.406 (m, 4H) 2.774-2.674 (m, 2H) 2.674-2.570 (m, 2H) 2.454 (t, J=7.7 Hz, 2H) 1.979-1.864 (m, 2H) 1.542-1.451 (m, 2H) 1.352 (dddd, J=12.8 Hz, 12.8 Hz, 12.8 Hz, 4.9 Hz, 1H) 1.294-1.158 (m, 10H) 0.840 (t, J=6.9 Hz, 3H).

### Example 23

### (−)-2-Amino-2-(6-octyl-1,2,3,4-tetrahydro-naphthalen-2-yl)-propane-1,3-diol (VIII-B)

The title compound was synthesized as per Example 21, (+)-2-Amino-2-(6-octyl-1,2,3,4-tetrahydro-naphthalen-2-yl)-propane-1,3-diol(ENANTIOMER 1), using (−)-2-Acetylamino-2-(6-octyl-1,2,3,4-tetrahydro-naphthalen-2-yl)-malonic acid diethyl ester (ENANTIOMER 2) as the diester starting material. MS: m/z=334.37 M+H; specific rotation−59.0 deg (0.1, ethanol); ¹H NMR (500 MHz, DMSO-d6) δ ppm 8.301 (s, 1H) 6.918 (d, J=7.8 Hz, 1H) 6.860 (d, J=8.2 Hz, 1H) 6.840 (s, 1H) 3.527-3.408 (m, 4H) 2.771-2.672 (m, 2H) 2.672-2.580 (m, 2H) 2.454 (t, J=7.6 Hz, 2H) 1.976-1.862 (m, 2H) 1.539-1.449 (m, 2H) 1.351 (dddd, J=12.4 Hz, 12.4 Hz, 12.4 Hz, 4.8 Hz, 1H) 1.296-1.159 (m, 10H) 0.840 (t, J=6.8 Hz, 3H).

### Example 24

### [2-Hydroxy-1-hydroxymethyl-1-(6-octyl-1,2,3,4-tetrahydro-naphthalen-2-yl)-ethyl]-carbamic acid benzyl ester (from ENANTIOMER 1)

(+)-2-Amino-2-(6-octyl-1,2,3,4-tetrahydro-naphthalen-2-yl)-propane-1,3-diol formate salt (ENANTIOMER 1) (397 mg, 1.05 mmol) and Potassium bicarbonate (314 mg, 3.14 mmol, Fisher) were dissolved in a 1-Neck round-bottom flask in Ethyl acetate (20 mL, Fisher) and Water (20 mL, Fisher). Benzyl chloroformate (164 μL, 1.15 mmol, Aldrich) was added to the mixture. The reaction mixture was stirred at room temperature for 1.5 hours. The organic layer was separated, washed with saturated sodium chloride, dried over sodium sulfate, filtered and evaporated. The product was purified by silica gel chromatography using 0-20% methanol in methylene chloride as eluent to provide the title compound in 450 mg yield (92%). MS: m/z=468.87 M+H.

### Example 25

### Phosphoric acid dibenzyl ester 4-(6-octyl-1,2,3,4-tetrahydro-naphthalen-2-yl)-2-oxo-oxazolidin-4-ylmethyl ester mixture of diastereoisomers

Tetrahexylammonium iodide (1.50 g, 3.11 mmol, Acros) was added to a solution of [2-hydroxy-1-hydroxymethyl-1-(6-octyl-1,2,3,4-tetrahydro-naphthalen-2-yl)-ethyl]-carbamic acid benzyl ester (ENANTIOMER 1; 718 mg, 1.54 mmol), tetrabenzyl pyrophosphate (1650 mg, 3.07 mmol, Aldrich) and silver(I) oxide (720 mg, 3.11 mmol, Aldrich) in Methylene chloride (32 mL, Acros). The reaction was stirred under an argon atmosphere under foil at room temperature for 3 days. The solids were filtered with a Whatman 0.45 um PTFE filter and the solvent was evaporated. The residue was purified by silica gel chromatography using 0-100% ethyl acetate in hexanes as eluent to give the product as a mixture of two diastereomers (Rf=0.27 in 1:1 ethyl acetate-hexanes, PMA visualization) in 416 mg yield (44%). MS: m/z=620.81 M+H.

### Example 26

### Phosphoric acid dibenzyl ester 4-(6-octyl-1,2,3,4-tetrahydro-naphthalen-2-yl)-2-oxo-oxazolidin-4-ylmethyl ester DIASTEREOMER 1 and Phosphoric acid dibenzyl ester 4-(6-octyl-1,2,3,4-tetrahydro-naphthalen-2-yl)-2-oxo-oxazolidin-4-ylmethyl ester DIASTEREOMER 2

Phosphoric acid dibenzyl ester 4-(6-octyl-1,2,3,4-tetrahydro-naphthalen-2-yl)-2-oxo-oxazolidin-4-ylmethyl ester mixture of diastereoisomers (0.40 g, 0.65 mmol) was separated using CHIRALPAK AZ column, with acetonitrile/methanol (75:25) as eluent. Two diastereoisomers were isolated: DIASTEREOMER 1 (166.3 mg, 42% yield, >99.9% ee) is characterized by analytical HPLC(CHIRALPAK AZ column, 4.6 mm ID×250 mm 75:25 acetonitrile/methanol 1 mL/min gives RT=5.558 min @ 210 nm). DIASTEREOMER 2 (203.7 mg, 51% yield, 99.8% ee) is characterized by analytical HPLC(CHIRALPAK AZ column, 4.6 mm ID×250 mm 75:25 acetonitrile/methanol 1 mL/min gives RT=8.137 min @ 210 nm).

### Example 27

### Phosphoric acid mono-[4-(6-octyl-1,2,3,4-tetrahydro-naphthalen-2-yl)-2-oxo-oxazolidin-4-ylmethyl]ester DIASTEREOMER 1

Phosphoric acid dibenzyl ester 4-(6-octyl-1,2,3,4-tetrahydro-naphthalen-2-yl)-2-oxo-oxazolidin-4-ylmethyl ester DIASTEREOMER 1 (166 mg, 0.268 mmol) was dissolved in methanol (10 mL, Fisher) in a round-bottom flask. 10% Palladium on carbon (28 mg, Aldrich) was added to the solution. The reaction was stirred at room temperature under a hydrogen (balloon pressure) atmosphere for 2 hours. The catalyst was filtered using a Whatman PTFE membrane filter. The solvent was concentrated and the product (107 mg, 91% yield) was used without further purification in Example 28.

### Example 28

### Phosphoric acid mono-[2-amino-3-hydroxy-2-(6-octyl-1,2,3,4-tetrahydro-naphthalen-2-yl)-propyl]ester DIASTEREOMER 1

Phosphoric acid mono-[4-(6-octyl-1,2,3,4-tetrahydro-naphthalen-2-yl)-2-oxo-oxazolidin-4-ylmethyl]ester (DIASTEREOMER 1; 107 mg, 0.243 mmol) was dissolved in ethanol (2.5 mL, Fisher) in a vial. Aqueous lithium hydroxide (4.2 M, 2.5 mL) was added to the solution and the reaction was heated at reflux overnight. The reaction was cooled and 11 mL of 1N HCl was added to neutralize. The reaction was evaporated. The residue was taken up in DMF and purified by preparative HPLC to provide the title compound in 49.4 mg yield (49%). MS: m/z=414.30 M+H; ¹H NMR (400 MHz, DMSO-d6) δ ppm 6.936 (d, J=7.9 Hz, 1H) 6.881 (d, J=8.9 Hz, 1H) 6.864 (s, 1H) 3.942-3.801 (m, 2H) 3.646-3.551 (m, 2H) 2.814-2.409 (m, 6H) 2.081-1.943 (m, 2H) 1.547-1.458 (m, 2H) 1.430 (m, 1H) 1.289-1.167 (m, 10H) 0.840 (t, J=6.9 Hz, 3H).

### Example 29

### Phosphoric acid mono-[2-amino-3-hydroxy-2-(6-octyl-1,2,3,4-tetrahydro-naphthalen-2-yl)-propyl]ester DIASTEREOMER 2

The title compound was synthesized as per Phosphoric acid mono-[2-amino-3-hydroxy-2-(6-octyl-1,2,3,4-tetrahydro-naphthalen-2-yl)-propyl]ester DIASTEREOMER 1 using Phosphoric acid dibenzyl ester 4-(6-octyl-1,2,3,4-tetrahydro-naphthalen-2-yl)-2-oxo-oxazolidin-4-ylmethyl ester DIASTEREOMER 2 as the phosphoric acid dibenzyl ester starting material. MS: m/z=414.43 M+H; ¹H NMR (400 MHz, DMSO-d6) δ ppm 6.916 (d, J=7.9 Hz, 1H) 6.872 (d, J=8.6 Hz, 1H) 6.852 (s, 1H) 3.900 (d, J=9.5 Hz, 2H) 3.582 (s, 2H) 2.811-2.622 (m, 4H) 2.468-2.416 (m, 2H) 2.062 (m, 1H) 1.971 (m, 1H) 1.551-1.452 (m, 2H) 1.387 (dddd, J=12.4 Hz, 12.4 Hz, 12.4 Hz, 5.0 Hz, 1H) 1.296-1.175 (m, 10H) 0.840 (t, J=7.0 Hz, 3H).

### Example 30

### Synthesis of diethyl 2-methyl-2-(6-octyl-1-oxo-1,2,3,4-tetrahydro-naphthalen-2-yl)malonate

Into a 1-Neck round-bottom flask was dissolved sodium hydride in mineral oil (60:40, Sodium hydride:Mineral Oil, 8.00 g; Aldrich) in N,N-Dimethylformamide (100 mL, 1.4 mol; Acros). The mixture was cooled at 0° C. and Methylpropanedioic acid, diethyl ester (25.24 mL, 0.1481 mol; Aldrich) in N,N-Dimethylformamide (100 mL, 1.4 mol; Acros) was added slowly. The reaction was stirred at 0° C. for 3 hours at which point a solution of 2-Bromo-6-octyl-3,4-dihydro-2H-naphthalen-1-one, 4, (20.00 g, 0.05930 mol; WUXI) (azeotroped with toluene) in N,N-Dimethylformamide (100 mL, 1.4 mol; Acros) was added. The residue was washed into the reaction with 10 mL of DMF. The reaction was stirred at room temperature for 24 hours. The mixture was poured into ice slowly, and was acidified to pH 3 with 1M HCl. The reaction was extracted with ethyl acetate, was washed with saturated sodium chloride, was dried with magnesium sulfate, was filtered and was concentrated. The residue was purified by silica gel chromatography using 0-30% ethyl acetate in hexanes as eluent to yield diethyl 2-methyl-2-(6-octyl-1-oxo-1,2,3,4-tetrahydronaphthalen-2-yl)malonate (17.63 g, 69.1% yield). LCMS (RT=2.58 min, m/z=431.72 [M+H]).

### Example 31

### Synthesis of diethyl 2-methyl-2-(6-octyl-1,2,3,4-tetrahydronaphthalen-2-yl)malonate

2-Methyl-2-(6-octyl-1-oxo-1,2,3,4-tetrahydro-naphthalen-2-yl)-malonic acid diethyl ester (22.70 g, 0.05272 mol) was dissolved in Methylene chloride (200 mL, 3 mol; Acros) and triethylsilane (33.7 mL, 0.211 mol; Aldrich) was added. The solution was cooled to 0° C. then titanium tetrachloride (23.2 mL, 0.211 mol; Aldrich) was added slowly. The reaction was allowed to stir 24 hours and LC/MS was taken which showed good conversion to the product. The reaction was quenched by slowly pouring into ice-cold saturated sodium bicarbonate. The reaction was then transferred to a separatory funnel and extracted with methylene chloride that was then dried and concentrated. The crude was then purified by combiflash using a 0-30 percent gradient to give diethyl 2-methyl-2-(6-octyl-1,2,3,4-tetrahydronaphthalen-2-yl)malonate (12.45 g, 56.7% yield). LCMS (RT=2.83 min, m/z=417.78 [M+H]).

### Example 32

### Synthesis of 3-ethoxy-2-methyl-2-(6-octyl-1,2,3,4-tetrahydronaphthalen-2-yl)-3-oxopropanoic acid

2-Methyl-2-(6-octyl-1,2,3,4-tetrahydro-naphthalen-2-yl)-malonic acid diethyl ester (6.50 g, 0.0156 mol) was dissolved in Ethanol (22.3 mL, 0.382 mol; Aldrich) and a solution of Potassium hydroxide (0.99 g, 0.018 mol; Fisher) in EtOH (2 ml) was added. The reaction was heated to 65° C. and stirred for 24 hours. The reaction was cooled down to room temperature and EtOH was removed under vacuum. 10 ml of water was added and acidified to pH1 with 1N HCl. The aqueous was then extracted with CHCl₃ and the organic layer was washed once with brine (the brine layer was acidified to pH1 make sure all solid dissolved in CHCl₃), and then dried with sodium sulfate. The concentrated residue was chromatographed with gradient EA/0.1% HOAc in HE(0-10%, 10-20%, 20-30%) to give desired acid 3-ethoxy-2-methyl-2-(6-octyl-1,2,3,4-tetrahydronaphthalen-2-yl)-3-oxopropanoic acid (3.13 g, 52% yield). LCMS (RT=2.47 min, m/z=389.35 [M+H]).

### Example 33

### Synthesis of ethyl 2-amino-2-(6-octyl-1,2,3,4-tetrahydronaphthalen-2-yl)propanoate

2-Methyl-2-(6-octyl-1,2,3,4-tetrahydro-naphthalen-2-yl)-malonic acid monoethyl ester (4.88 g, 0.0126 mol) was dissolved in Toluene (126 mL, 1.18 mol) and Triethylamine (2.1 mL, 0.015 mol; Aldrich) and Diphenylphosphonic azide (2.7 mL, 0.012 mol) were added and was heated to reflux for 2.5 hours. The reaction was cooled to at 0° C. and was added 1 M of Sodium Trimethylsilanolate in THF (25 mL) and was stirred for 1 hour at 25° C. The reaction was quenched with 5% citric acid, was evaporated to remove THF and toluene, and washed with Et₂O (2×). The remained aqueous solution was basified to pH13 with 1N NaOH, extracted with CH₂Cl₂. The combined CH₂Cl₂ solution was washed with brine, dried, and concentrated. The residue was chromatographed with ISCO combiflash (EtOAc:Hexane(0.1% TEA) 0-70%) to give ethyl 2-amino-2-(6-octyl-1,2,3,4-tetrahydronaphthalen-2-yl)propanoate as a mixture of diastereomers (3.20 g, 70.9% yield, LCMS RT=1.83 min, m/z=360.67 [M+H]).

The diastereomers of ethyl 2-amino-2-(6-octyl-1,2,3,4-tetrahydronaphthalen-2-yl)propanoate (15) (0.96 g) were separated in two stages. The first stage used CHIRALPAK AD-H (4.6 mm ID×250 mm) with ACN/MeOH (75/25) as eluent at room temperature under UV 230 nm detection. Two diastereomers were isolated: Isomer 15a (0.215 g, 98% d.e., 98% yield, room temperature=10.947 min), LCMS room temperature=1.83 min, m/z=360 [M+1]; Isomer 15b (0.238 g, 97.6% d.e. 94.4% yield, room temperature=8.726 min), LCMS room temperature=1.78 min, m/z=360 [M+1]; and mixture of 15c and 15d (0.456 g, 93.4% yield, room temperature=5.707 min). The second stage was using CHIRALPAK AY (4.6 mm ID×250 mm) with Hex/IPA (85/15) as eluent at room temperature under UV 230 nm detection. From 0.389 g of the above mixture, the other two diastereomers were isolated: Isomer 15c (0.143 g, 99% d.e., 88.5% yield, room temperature=4.511 min), LCMS room temperature=1.78 min, m/z=360 [M+1]; Isomer 15d (0.175 g, 96.4% d.e. 79% yield, room temperature=5.919 min), LCMS room temperature=1.78 min, m/z=360 [M+1].

### Example 34

### Synthesis of (R)-2-amino-2-((R)-6-octyl-1,2,3,4-tetrahydronaphthalen-2-yl)propan-1-ol isomer 1 (16a)

Into a 1-Neck round-bottom flask was added 15a (215.0 mg, 0.0005980 mol), Tetrahydrofuran (6.0 mL, 0.074 mol; Acros) was added followed by 1 M of Lithium tetrahydroaluminate in THF (1.8 mL). The mixture was heated to reflux for 2 hours. The reaction mixture was cooled at 0° C. and quenched with saturated Rochelle salt solution. The mixture was extracted with EtOAc (3×5 ml) and was washed with brine and was dried over sodium sulfate. TLC monitoring showed no starting material left. LCMS gave a single peak. The concentrated residue was chromatographed with MeOH/CH₂Cl₂ (0-50%) to give 161.2 mg (84.9% yield) 16a as a white powder. LCMS RT=1.68 min, m/z=318.67 [M+1]. ¹H NMR (CD₃OD, 400 MHZ) 0.90 (t, J=6.8 Hz, 3H), 1.19 (s, 3H), 1.31 (m, 11H), 1.49 (dd, J=12.6, 5.2 Hz, 1H), 1.59 (dd, J=12.6, 5.8 Hz, 1H), 2.04 (m, 2H), 2.52 (t, 7.7 Hz, 2H), 2.62 (dd, J=15.5, 12.6 Hz, 1H), 2.75-2.90 (m, 3H), 3.54 (d, J=11.3 Hz, 1H), 3.69 (d, J=11.3 Hz, 1H), 6.89 (s, 1H), 6.90 (d, J=7.6 Hz, 1H), 6.98 (d, J=7.6 Hz, 1H). The stereochemistry was determined by comparison with enantiomers of 2-Amino-2-(6-octyl-1,2,3,4-tetrahydro-naphthalen-2-yl)-propane-1,3-diol.

### Example 35

### Synthesis of (R)-2-amino-2-((S)-6-octyl-1,2,3,4-tetrahydronaphthalen-2-yl)propan-1-ol, isomer 2 (16b)

Into a 1-Neck round-bottom flask was added 15b (240.4 mg, 0.0006619 mol), Tetrahydrofuran (6.0 mL, 0.074 mol; Acros) was added followed by 1 M of Lithium tetrahydroaluminate in THF (1.8 mL). The mixture was heated to reflux for 2 hours. The reaction mixture was cooled at 0° C. and quenched with saturated Rochelle salt solution. The mixture was extracted with EtOAc (3×5 mL) and was washed with brine and was dried over sodium sulfate. TLC monitoring shows no starting material left. LCMS gave a single peak. The concentrated residue was chromatographed with MeOH/CH₂Cl₂ (0-50%) to give 150.8 mg (71.8% yield) 16b as a white powder. LCMS RT=1.68 min, m/z=318.67 [M+1]. 1H NMR (CD₃OD, 400 MHZ) 0.90 (t, J=7.0 Hz, 3H), 1.17 (s, 3H), 1.31 (m, 11H), 1.44 (m, 1H), 1.58 (m, 1H), 2.00 (m, 2H), 2.52 (t, 7.6 Hz, 2H), 2.66 (dd, J=15.8, 12.4 Hz, 1H), 2.74-2.92 (m, 3H), 3.54 (d, J=11.2 Hz, 1H), 3.63 (d, J=11.2 Hz, 1H), 6.87 (s, 1H), 6.89 (d, J=7.7 Hz, 1H), 7.00 (d, J=7.7 Hz, 1H). The stereochemistry was determined by comparison with enantiomers of 2-Amino-2-(6-octyl-1,2,3,4-tetrahydro-naphthalen-2-yl)-propane-1,3-diol.

### Example 36

### Synthesis of (S)-2-amino-2-((S)-6-octyl-1,2,3,4-tetrahydronaphthalen-2-yl)propan-1-ol, isomer 3 (16c)

Into a 1-Neck round-bottom flask was added 15c (120.2 mg, 0.0003310 mol), Tetrahydrofuran (3.3 mL, 0.041 mol; Acros) was added followed by 1 M of Lithium tetrahydroaluminate in THF (0.99 mL). The mixture was heated to reflux for 2 hours. The reaction mixture was cooled at 0° C. and quenched with saturated Rochelle salt solution. The mixture was extracted with EtOAc (3×5 mL) and was washed with brine and was dried over sodium sulfate. TLC monitoring showed no starting material left. LCMS gave a single peak. The concentrated residue was chromatographed with MeOH/CH₂Cl₂ (0-50%) to give 43.0 mg (40.9% yield) 16c as a white powder. LCMS RT=1.68 min, m/z=318.67 [M+1]. ¹H NMR (CD₃OD, 400 MHZ) is the same as 16a, shows characteristic peak 3.54 (d, J=11.3 Hz, 1H), 3.69 (d, J=11.3 Hz, 1H). It was identified as the enantiomer of 16a.

### Example 37

### Synthesis of (S)-2-amino-2-((R)-6-octyl-1,2,3,4-tetrahydronaphthalen-2-yl)propan-1-ol, isomer 4 (16d)

Into a 1-Neck round-bottom flask was added 15d (175.0 mg, 0.0004818 mol), Tetrahydrofuran (4.9 mL, 0.060 mol; Acros) was added followed by 1 M of Lithium tetrahydroaluminate in THF (1.4 mL). The mixture was heated to reflux for 2 hours. The reaction mixture was cooled at 0° C. and quenched with saturated Rochelle salt solution. The mixture was extracted with EtOAc (3×5 ml) and was washed with brine and was dried over sodium sulfate. TLC monitoring showed no starting material left. LCMS gave a single peak. The concentrated residue was chromatographed with MeOH/CH₂Cl₂ (0-50%) to give 77.4 mg (50.6% yield) 16d as a white powder. LCMS RT=1.68 min, m/z=318.67 [M+1]. ¹H NMR (CD₃OD, 400 MHZ) is the same as 16b, shows characteristic peak 3.54 (d, J=11.2 Hz, 1H), 3.63 (d, J=11.2 Hz, 1H). It was identified as the enantiomer of 16b.

### Example 38

### Synthesis of tert-butyl (R)-1-hydroxy-2-((R)-6-octyl-1,2,3,4-tetrahydronaphthalen-2-yl)propan-2-ylcarbamate (17)

2-Amino-2-(6-octyl-1,2,3,4-tetrahydro-naphthalen-2-yl)-propan-1-ol (16a) (40.7 mg, 0.000128 mol) was dissolved in Chloroform (2.3 mL, 0.029 mol) and Saturated Aqueous Sodium Bicarbonate Solution (1.5 mL, 0.015 mol) and Di-tert-Butyldicarbonate (33.6 mg, 0.000154 mol) was added and the mixture was stirred at room temperature for 24 h. TLC showed complete reaction. After separation of organic layer, the aqueous layer was extracted with CHCl₃. The organic layer was washed with brine and dried over anhydrous Na₂SO₄. The concentrated residue was chromatographed with MeOH/CH₂Cl₂ (0-55%) to give 17 (45.8 mg, 85.5% yield) as a white solid. ¹H NMR showed the identity of the compound.

### Example 39

### Synthesis of (R)-[1-Methyl-1-((R)-6-octyl-1,2,3,4-tetrahydro-naphthalen-2-yl)-2-(1,5-dihydrobenzo[e][1,3,2]dioxaphosphepin-3-yloxy)-ethyl]-carbamic acid tert-butyl Ester (18)

To a solution of [2-Hydroxy-1-methyl-1-(6-octyl-1,2,3,4-tetrahydro-naphthalen-2-yl)-ethyl]-carbamic acid tert-butyl ester (17) (44.8 mg, 0.000107 mol) and 1H-Tetrazole (22.6 mg, 0.000323 mol) in Tetrahydrofuran (1.1 mL, 0.014 mol) was added o-Xylylene N,N-diethylphosphoramidite (34.8 uL, 0.000161 mol) at room temperature. The resulting mixture was stirred at room temperature for overnight; TLC showed no starting material left. Hydrogen peroxide (240 uL, 0.0024 mol) was added and the mixture was stirred at room temperature for 1 hour. The reaction was quenched with saturated NaS₂O₃, then extracted with EtOAc, then dried over Na₂SO₄. The residue was chromatographed with MeOH/CH₂Cl₂ (0-100%) to give 18 (47.3 mg, 73.5% yield). ¹H NMR showed the identity of the compound.

### Example 40

### Synthesis of tert-butyl (R)-2-((R)-6-octyl-1,2,3,4-tetrahydronaphthalen-2-yl)-1-(phosphonooxy)propan-2-ylcarbamate (19)

[1-Methyl-1-(6-octyl-1,2,3,4-tetrahydro-naphthalen-2-yl)-2-(1,5-dihydrobenzo [e][1,3,2]dioxaphosphepin-3-yloxy)-ethyl]-carbamic acid tert-butyl ester 18 (47.3 mg, 0.0000789 mol) was dissolved in Methanol (1.0 mL, 0.025 mol) and was added 10% Palladium on Carbon (1:9, Palladium:carbon black, 4.8 mg). The mixture was stirred under Hydrogen (2 L, 0.07 mol) for 2 h, filtered through celite and was washed with MeOH. The concentrated residue was dissolved in CH₂Cl₂ and was chromatographed with MeOH/CH₂Cl₂ (0-50%) to give 19 (23.2 mg, 59.1% yield). ¹H NMR showed the identity of the compound.

### Example 41

### Synthesis of (R)-2-amino-2-((R)-6-octyl-1,2,3,4-tetrahydronaphthalen-2-yl)propyl dihydrogen phosphate (110)

[tert-butyl (R)-2-((R)-6-octyl-1,2,3,4-tetrahydronaphthalen-2-yl)-1-(phosphonooxy)propan-2-ylcarbamate (19) (23.2 mg, 0.0000466 mol) was dissolved in acetic acid (2.0 mL, 0.034 mol) and 10 M of hydrogen chloride in water (0.5 mL) was added and the mixture was stirred for 1 d. Lypholyzing gave 110 as a white solid (16.0 mg, 86.3% yield). LCMS gave a single peak room temperature=1.58 min, m/z=398, [M]⁺. ¹H NMR (CD₃OD, 400 MHZ) 0.89 (t, J=7.0 Hz, 3H), 1.32 (s, 3H), 1.24-1.36 (m, 14H), 1.52-1.56 (m, 1H), 2.52 (t, 7.6 Hz, 2H), 2.65 (dd, J=15.5, 12.0 Hz, 1H), 2.78-2.94 (m, 3H), 3.93 (d, J=11.2, 3.8 Hz, 1H), 4.11 (dd, J=11.2, 4.5 Hz, 1H), 6.88 (s, 1H), 6.90 (d, J=7.4 Hz, 1H), 6.99 (d, J=7.4 Hz, 1H).

### Example 42

### 1,3-Dihydroxy-2-[(2S)-6-octyl-1,2,3,4-tetrahydronaphthalen-2-yl]propan-2-aminium Bromide Monohydrate

(−)-2-Amino-2-(6-octyl-1,2,3,4-tetrahydro-naphthalen-2-yl)-propane-1,3-diol (50.0 mg, 0.000150 mol) was dissolved partially in methanol (5.0 mL, 0.12 mol). To the filtrated solution was added hydrogen bromide (5.0 mL, 0.046 mol; 48% solution) slowly, and the precipitate was gradually formed after the first 1 ml of HBr. After 2 h the solid was filtered out and washed with water and Et₂O. Lyophylizing gave 40.2 mg white needles. M.P. 158-159° C. LCMS gave a single peak RT=1.63 min (M/Z=335, parent [M+1]+). X-ray showed the compound to be the HBr salt monohydrate with a configuration (2S) (below).

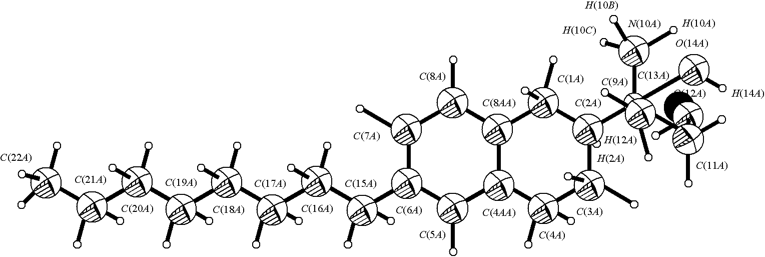

Structure of 1,3-Dihydroxy-2-[(2S)-6-octyl-1,2,3,4-tetrahydronaphthalen-2-yl]propan-2-aminium Bromide Monohydrate at 193(2)K

### Example 43

### X-ray Experimental of 1,3-Dihydroxy-2-[(2S)-6-octyl-1,2,3,4-tetrahydronaphthalen-2-yl]propan-2-aminium Bromide Monohydrate

The compound was provided as tiny, colorless, thin needles grown as above. A needle 0.002 mm×0.025 mm×0.125 mm in size was selected, mounted on a nylon loop with Paratone-N oil, and transferred to a Bruker SMART APEX II diffractometer equipped with an Oxford Cryosystems 700 Series Cryostream Cooler and Mo Kα radiation (λ=0.71073 Å). The diffraction from the needle was too weak. Therefore, only a preliminary study with a partial data set (91.8% complete to 1.1 Å resolution) was targeted. The crystal did not exhibit any significant data beyond 1.1 Å resolution, so a standard 0.76961 Å resolution (θₘₐₓ=27.5°) data set was unwarranted and was not pursued. A total of 585 frames were collected at 193 (2) K to θₘₐₓ=18.85° with an ω oscillation range of 0.5°/frame, and an exposure time of 90 s/frame using the APEX2 suite of software. (Bruker AXS, 2006a) Unit cell refinement on all observed reflections, and data reduction with corrections for Lp and decay were performed using SAINT. (Bruker AXS, 2006b) Scaling and a numerical absorption correction were done using SADABS. (Bruker AXS, 2004) The minimum and maximum transmission factors were 0.85628 and 0.96587, respectively. A total of 3188 reflections were collected, 2757 were unique (Rᵢₙₜ=0.0378), and 1982 had I>2σ(I). A lack of systematic absences suggested that the compound had crystallized in the triclinic space group P1 (No. 1). The observed mean |E²−1| value was 0.718 (versus the expectation values of 0.968 and 0.736 for centric and noncentric data, respectively).

The structure was solved by direct methods and refined by full-matrix least-squares on F² using SHELXTL. (Bruker AXS, 2001) The asymmetric unit was found to contain two aminium cations, two bromide anions and two water molecules. Due to the low resolution of the data available, only the two bromine atoms were refined with anisotropic displacement coefficients. All other non-hydrogen atoms were refined isotropically. The hydrogen atoms were assigned isotropic displacement coefficients U(H)=1.2U(C), 1.5U(C_{methyl})_{,} 1.5U(N) or 1.5U(O), and their coordinates were allowed to ride on the atoms to which they were attached. The hydrogen atoms for the water molecules were not observed and were not included in the cycles of least-squares. The refinement converged to R(F)=0.0554, wR(F²)=0.0927, and S=0.958 for 1982 reflections with I>2σ(I), and R(F)=0.0929, wR(F²)=0.1045, and S=0.958 for 2757 unique reflections, 225 parameters and 3 origin-defining restraints. The maximum |Δ/σ| in the final cycle of least-squares was less than 0.001, and the residual peaks on the final difference-Fourier map ranged from −0.302 to 0.384 eÅ⁻³. Scattering factors were taken from the International Tables for Crystallography, Volume C. (Maslen et al., 1992, and Creagh & McAuley, 1992)

The Flack parameter refined to 0.03 (3) [versus the expectation values of 0 for the correct hand and 1 for the wrong hand] indicating that the coordinates given below are for the correct hand of the molecular cations. Thus, the absolute configuration is (2S) at the chiral center in each of the two crystallographically independent cations, and the compound is unequivocally determined to be 1,3-Dihydroxy-2-[(2S)-6-octyl-1,2,3,4-tetrahydronaphthalen-2-yl]propan-2-aminium Bromide Monohydrate by anomalous dispersion methods. (Flack, 1983)

### Example 44

### Sphingosine Kinase Assay

Recombinant sphingosine kinase type 2 (SPHK2) is prepared by forcing the expression of the mouse or human recombinant enzyme by transfecting the relevant plasmid DNA into HEK293T cells. After about 60 hours, cells are harvested, broken and the non-microsomal (e.g., soluble) fraction is retained. The broken cell supernatant fluid containing the recombinant enzyme is mixed with test compounds (e.g., FTY-720, AA151, VIII and XVIII) (5-50 micromolar) and γ-³²P-ATP and incubated for 0.5-2.0 hours at 37° C. The lipids in the reaction mixture are extracted into an organic solvent and displayed by normal phase thin layer chromatography. The radio-labeled bands are detected by autoradiography, scraped from the plate and quantified by scintillation counting.

### Example 45

### GTPγS-35 binding Assay

This assay illustrates agonist activation of G protein coupled receptors (GPCRs) in isolation. The assay forces expression concomitantly of a recombinant GPCR (e.g., the S1P₁-5 receptor) and each of the three subunits (typically, α-2, β-1, or γ-2) of a heterotrimeric G protein in a HEK293T cell by transfecting the cell with four plasmid DNAs encoding the respective proteins. About 60 hours after transfection the cells are harvested, opened, and the nucleus discarded. The crude microsome is prepared from the remainder. Agonist (e.g., S1P) stimulation of the receptor-G protein complex on the microsomes results in the exchange of GTP for GDP on the α-subunit in a dose-dependent manner. The GTP-bound α-subunit is detected using a GTP analog (GTPγS-35), which is a radionuclide (sulfur-35) labeled phosphothionate that is not hydrolyzed to GDP. The microsomes with the adherent G proteins are collected by filtration and the bound GTPγS-35 quantified in a liquid scintillation counter. The assay yields relative potency (EC₅₀ values) and maximum effect (efficacy, Eₘₐₓ). Antagonist activity is detected as rightward shifts in the agonist dose-response curve in the presence of a fixed amount of antagonist. If the antagonist behaves competitively, the affinity of the receptor/antagonist pair (Kᵢ) can be determined.

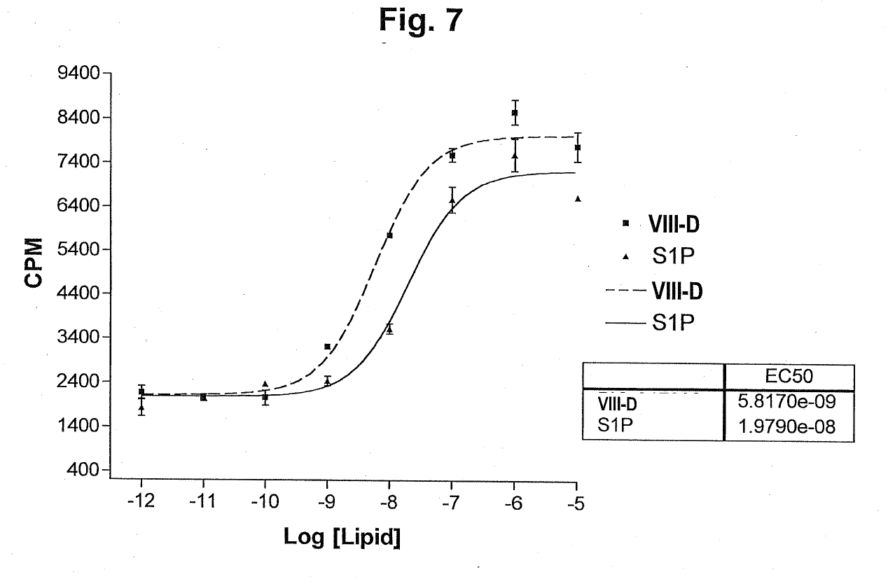

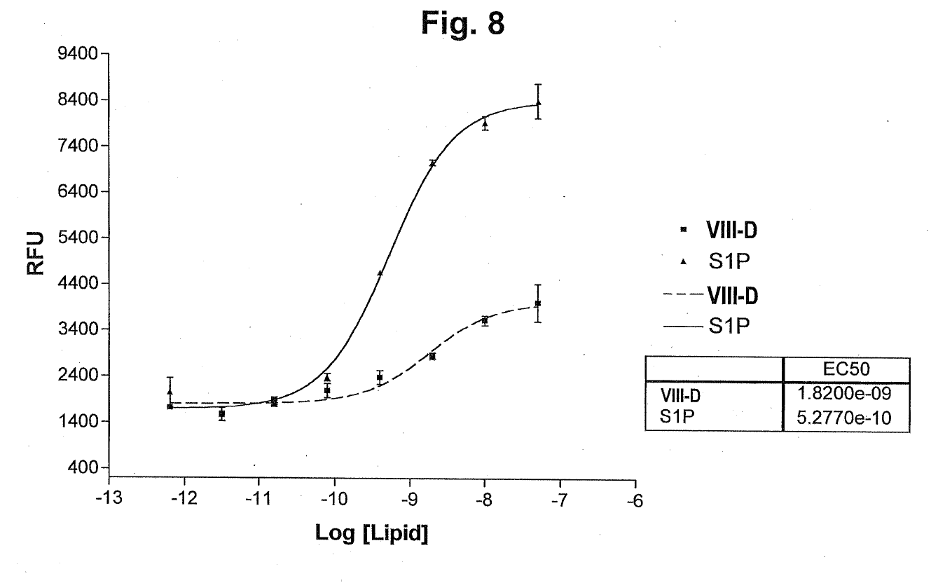

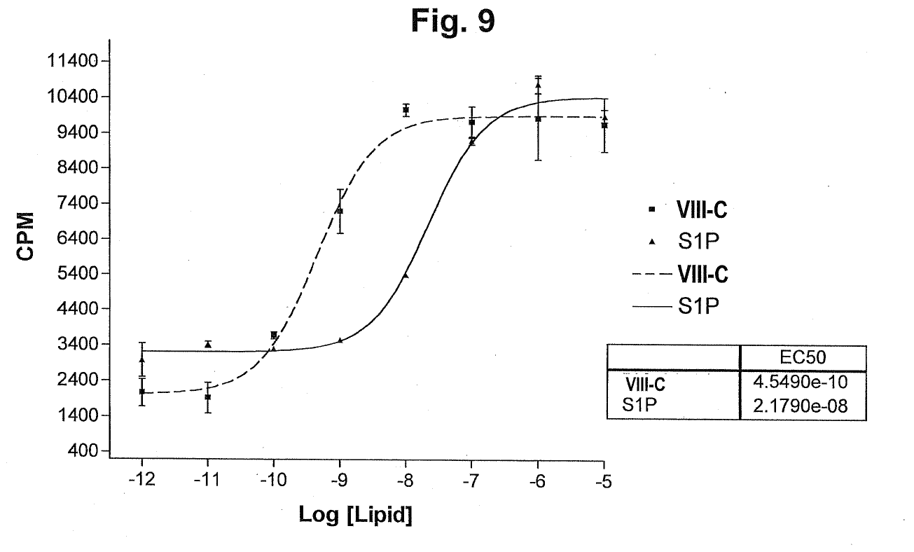

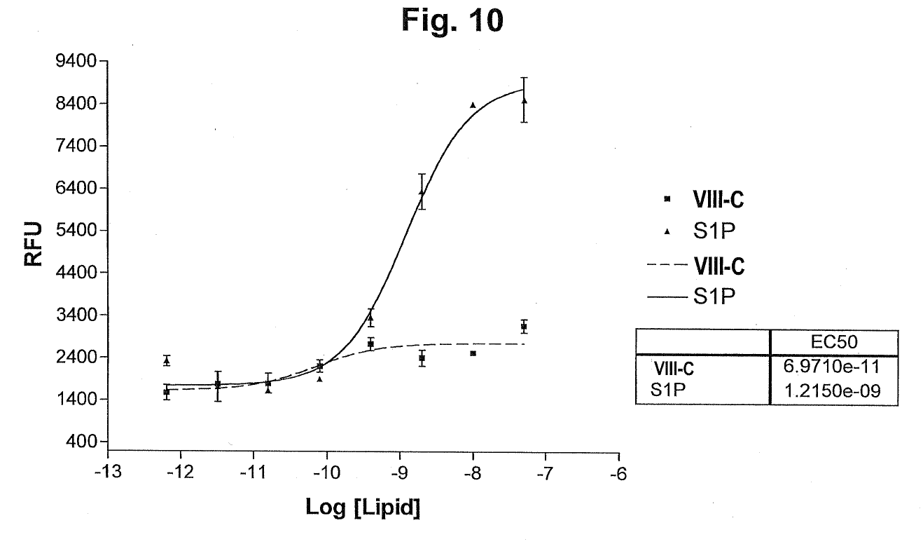

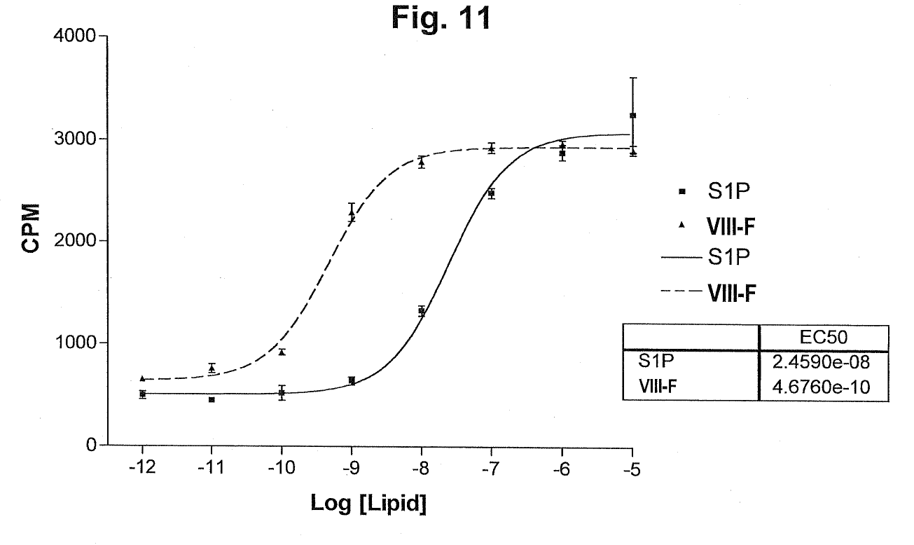

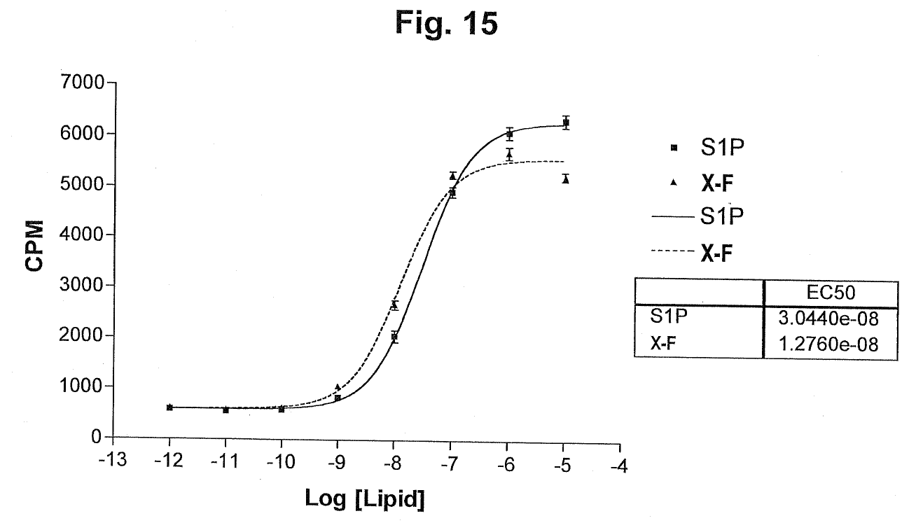

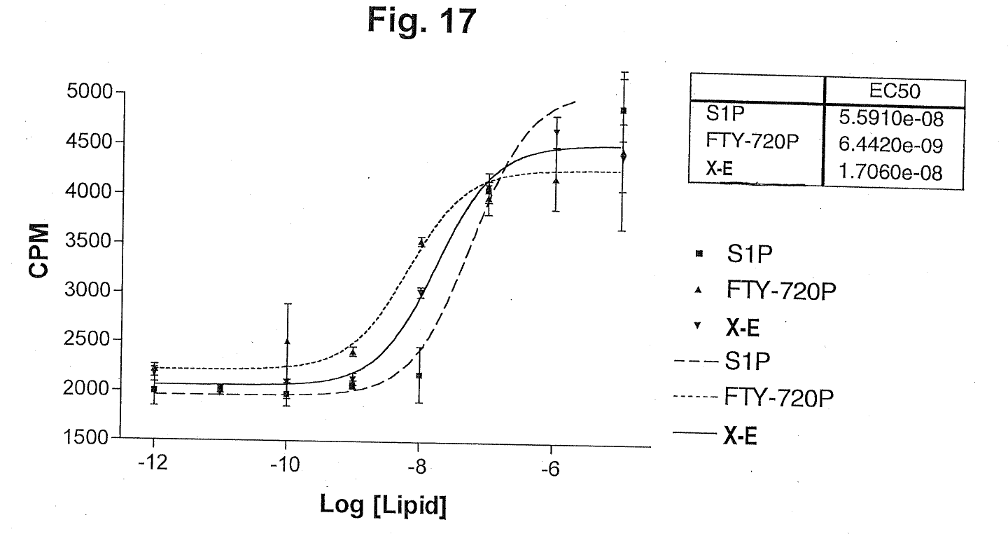

The phosphorylated forms of compounds VIII-A and VIII-B, compounds VIII-C and VIII-D, are low potency, partial agonists at the S1P₃ receptor (See FIG. 8 and FIG. 10). Compounds VIII-C, VIII-D, VIII-E, VIII-F and X-E are more potent at S1P, and less potent at S1P₃, relative to S1P (See FIGS. 7, 9, 11, 12, 15 and 17). The assay was performed as described in Davis, M. D., J. J. Clemens, T. L. Macdonald and K. R. Lynch (2005) “S1P Analogs as Receptor Antagonists” Journal of Biological Chemistry, vol. 280, pp. 9833-9841.

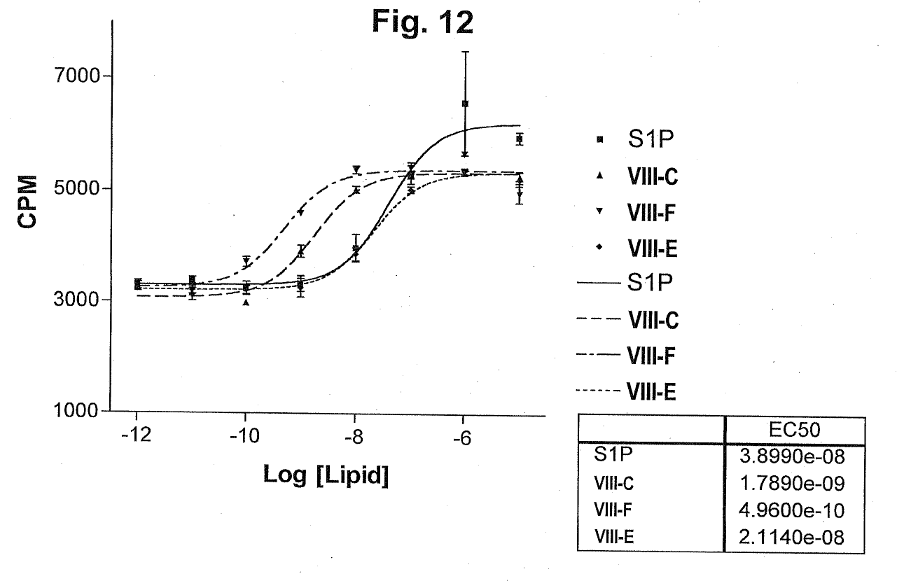

The phosphorylated forms of compounds VIII(+) (VIII-C; a mixture of monophosphorylated isomers), VIII(−) (VIII-D; a mixture of monophosphorylated isomers), compound VIII-F and separated compound VIII-E were tested in the Davis assay. The results are illustrated in FIG. 12.

### Example 46

### Lymphopenia Assay with Stereoisomers

The stereospecific compounds VIII-A and VIII-B were evaluated as described above in the lymphopenia assay utilizing 8 doses 3 mice per dose where compound VIII-A was found to have an ED₅₀ of 0.2 mg/kg and compound VIII-B was found to have an ED₅₀ of 2 mg/kg.

### Example 47

### Lymphopenia Assay

Compounds (e.g., primary alcohols such as compound VIII) are dissolved in 2% hydroxypropyl beta-cyclodextrin and introduced into groups of mice by oral gavage at doses from 0.01, 1.0 and 10 mg/kg body weight. After 24 hours and 48 hours, the mice are lightly anesthetized and ca. 0.1 ml of blood is drawn from the orbital sinus. The number of lymphocytes (in thousands per microliter of blood; normal is 4-11) is determined using a Hemavet blood analyzer. There are three mice/group, the strain was mixed sv129×C57BL/6. Active compounds (e.g., compound VIII-C and compound VIII-D) are dissolved in acidified DMSO at 20 mM, and diluted 1:20 into 2% hydroxypropyl beta-cyclodextrin in water with mixing. This solution is introduced into mice by intraperitoneal (i.p.) injection at doses of 0.01, 1.0 and 10 mg/kg body weight.

### Example 48

### Calcium Mobilization

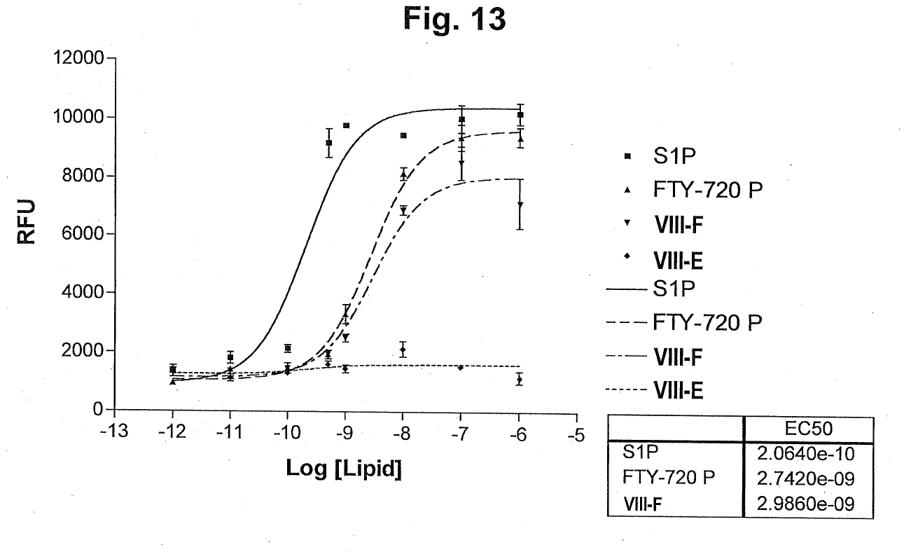

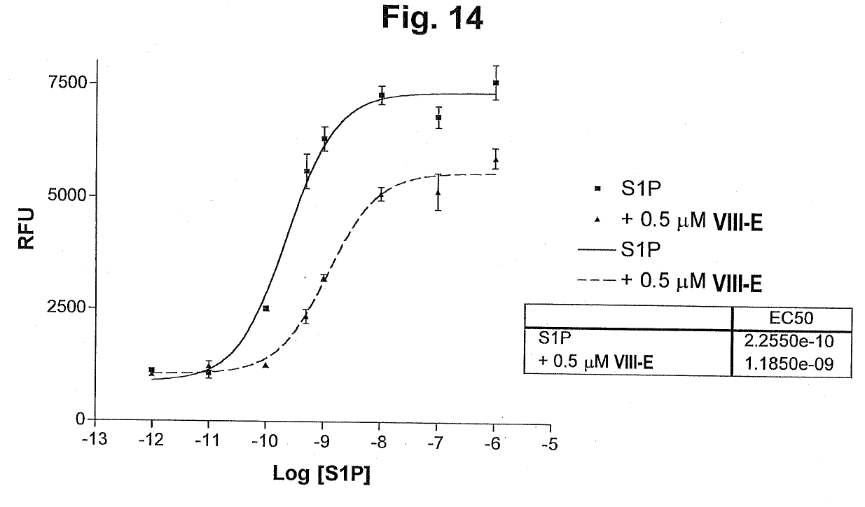

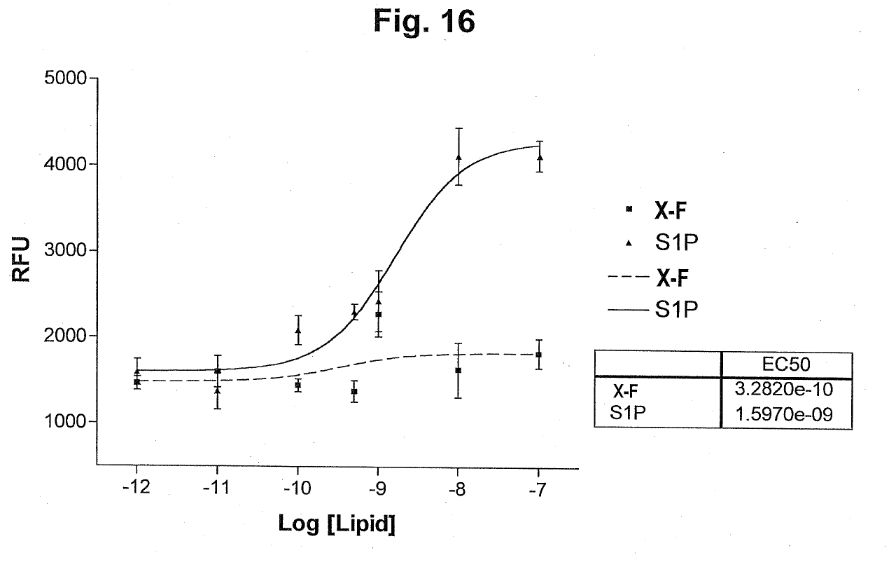

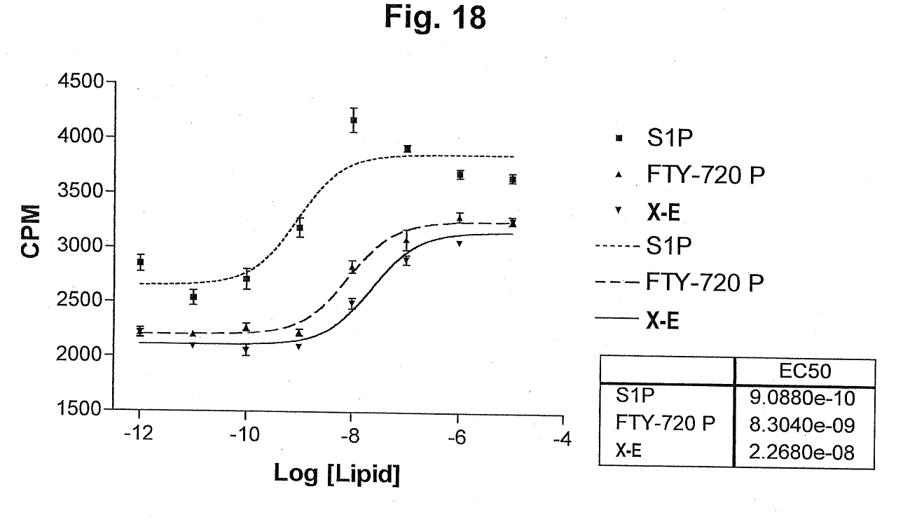

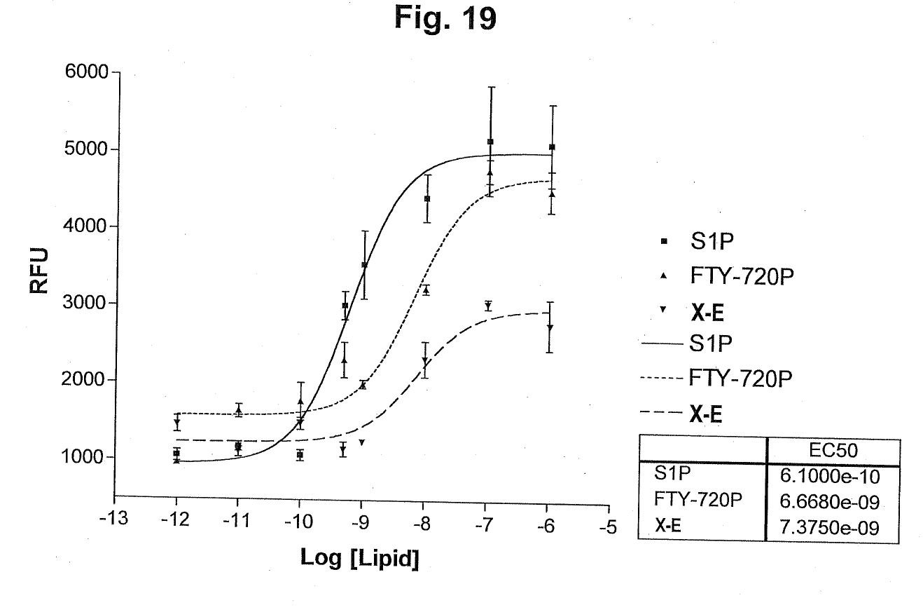

The disclosed compounds were tested in a calcium mobilization assay to determine agonist and antagonist activity at the human S1P₃ receptor. The procedure is as described in Davis et al. (2005) Journal of Biological Chemistry, vol. 280, pp. 9833-9841. The test compounds VIII-C, VIII-D, VIII-E, VIII-F, phosphorylated FTY-720 (FTY-720 P) X-E and X-F were tested alone (e.g., agonist activity) and some were collided with sphingosine 1-phosphate (antagonist activity). The results are illustrated in FIGS. 8, 10, 13, 14, 16, 18 and 19.

The invention should not be construed to be limited solely to the assays and methods described herein, but should be construed to include other methods and assays as well. Other methods which were used but not described herein are well known and within the competence of one of ordinary skill in the art of chemistry, biochemistry, molecular biology, and clinical medicine. One of ordinary skill in the art will know that other assays and methods are available to perform the procedures described herein.

The abbreviations used herein have their conventional meaning within the clinical, chemical, and biological arts. In the case of any inconsistencies, the present disclosure, including any definitions therein will prevail.

The invention should not be construed to be limited solely to the assays and methods described herein, but should be construed to include other methods and assays as well. Other methods which were used but not described herein are well known and within the competence of one of ordinary skill in the art of chemistry, biochemistry, molecular biology, and clinical medicine. One of ordinary skill in the art will know that other assays and methods are available to perform the procedures described herein.

The abbreviations used herein have their conventional meaning within the clinical, chemical, and biological arts. In the case of any inconsistencies, the present disclosure, including any definitions therein will prevail.

The disclosures of each and every patent, patent application, and publication cited herein are expressly incorporated herein by reference in their entirety into this disclosure. Illustrative embodiments of this disclosure are discussed and reference has been made to possible variations within the scope of this disclosure. These and other variations and modifications in the disclosure will be apparent to those skilled in the art without departing from the scope of the disclosure, and it should be understood that this disclosure and the claims shown below are not limited to the illustrative embodiments set forth herein.

1. An enantiomeric ally pure compound of formula I: 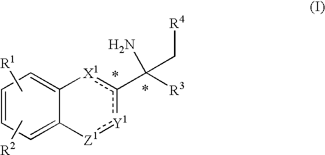
wherein X¹, Y¹ and Z¹ are independently O, CR^{a}, CR^{a}R^{b}, N, NR^{c}, or S;
R¹ and R² are independently hydrogen, halo, halo(C₁-C₁₀)alkyl, cyano, —NR^{a}R^{b}, (C₁-C₂₀)alkyl, (C₂-C₂₀)alkenyl, (C₂-C₂₀)alkynyl, (C₁-C₂₀)alkoxy, (C2-C₂₆)alkoxyalkyl, (C₃-C₁₂)cycloalkyl, (C₆-C₁₀)aryl, (C₇-C₃₀)arylalkyl, (C₂-C₁₀)heterocyclic, (C₄-C₁₀)heteroaryl, or (C₄-C₁₀)heteroaryl(C₁-C₂₀)alkyl; or
R² can be a group having formula II, III, IV, V, or VI; 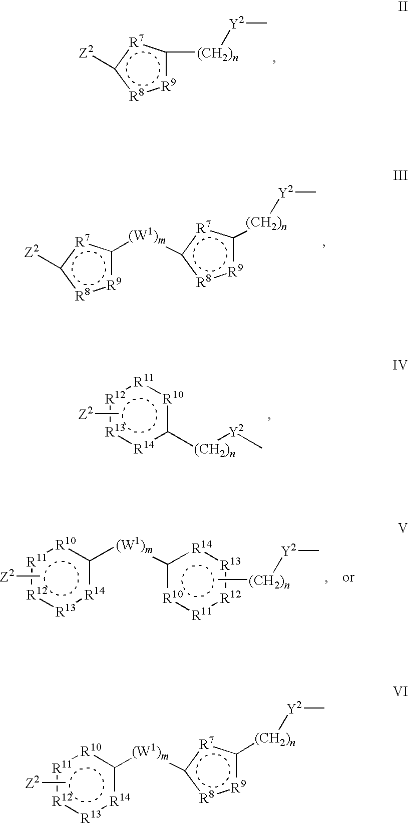
R⁷, R⁸, R⁹, R¹⁰, R¹¹, R¹², R¹³, and R¹⁴ are independently O, S, C, CR¹⁵, CR¹⁶R¹⁷, C═O, N or NR¹⁸;
R¹⁵, R¹⁶ and R¹⁷ are independently hydrogen, halo, (C₁-C₁₀)alkyl, (C₁-C₁₀)alkyl substituted with halo, hydroxy, (C₁-C₁₀)alkoxy, or cyano; and where R¹⁸ can be hydrogen or (C₁-C₁₀)alkyl;
where Z² is hydrogen, halo, halo(C₁-C₁₀)alkyl, cyano, —NR^{a}R^{b}, (C₁-C₂₀)alkyl, (C₂-C₂₀)alkenyl, (C₂-C₂₀)alkynyl, (C₁-C₂₀)alkoxy, (C₂-C₂₆)alkoxyalkyl, (C₃-C₁₂)cycloalkyl, (C₆-C₁₀)aryl, (C₇-C₃₀)arylalkyl, (C₂-C₁₀)heterocyclic, (C₄-C₁₀)heteroaryl, or (C₄-C₁₀)heteroaryl(C₁-C₂₀)alkyl; wherein the alkyl, alkenyl, alkynyl, cycloalkyl, aryl, heterocyclic, or heteroaryl groups of Z² are optionally perfluorinated or optionally substituted with 1, 2, 3, or 4 groups where the substituent groups are independently hydroxy, halo, cyano, (C₁-C₁₀)alkoxy, C₆-aryl, (C₇-C₂₄)arylalkyl, oxo (═O), or imino (═NR^{d}), wherein one or more of the carbon atoms in the Z² alkyl groups can be independently replaced with non-peroxide oxygen, sulfur or NR^{c}; [symbol]
indicates one or more optional double bonds;
wherein Y² is a bond, O, S, C═O, or NR^{c}, CH₂; W¹ is a bond; —CH₂— and m is 1, 2, or 3, or (C═O)(CH₂)₁₋₅ and m is 1; wherein W¹ is optionally interrupted with non-peroxide O, S, C═O, or NR^{c};
each [symbol] represents an optional double bond; and n is 0, 1, 2, or 3;
R³ is hydrogen, (C₁-C₁₀)alkyl, hydroxy(C₁-C₁₀)alkyl or (C₁-C₁₀)alkoxy; and
R⁴ is hydroxyl (—OH), phosphate (—OPO₃H₂), phosphonate (—CH₂PO₃H₂), or alpha-substituted phosphonate;
R^{a}, R^{b}, and R^{c} are independently hydrogen, or (C₁-C₁₀)alkyl;
wherein the
alkyl, alkenyl, alkynyl, cycloalkyl, aryl, heterocyclic, or heteroaryl groups of R¹ and R² independently are optionally perfluorinated or optionally substituted with 1, 2, 3, or 4 groups where the substituent groups are independently hydroxy, halo, cyano, (C₁-C₁₀)alkoxy, C₆-aryl, (C₇-C₂₄)arylalkyl, oxo (═O), or imino (═NR^{d}), wherein one or more of the carbon atoms in the R¹ or R² alkyl groups can be independently replaced with non-peroxide oxygen, sulfur or NR^{c}; the alkyl groups of R³ are optionally substituted with 1, or 2 hydroxy groups; and R^{d} is hydrogen, or (C₁-C₁₀)alkyl; or
a pharmaceutically acceptable salt or ester thereof.

2. The compound of claim 1, wherein the configuration is RR, RS, SR or SS.

3. The compound of claim 1, wherein R¹ is hydrogen, fluorine, chlorine, bromine, trifluoromethyl, methoxy, (C₁-C₆)alkyl, (C₁-C₆)haloalkyl, (C₁-C₆)alkyl substituted with, alkoxy or cyano, alkyl-substituted aryl, aryl-substituted alkyl, or aryl-substituted arylalkyl.

4. The compound of claim 3, wherein R¹ is hydrogen, trifluoromethyl, or —CH₂CF₃.

5. The compound of claim 3, wherein R¹ is benzyl, phenylethyl, or methyl benzyl.

6. The compound of claim 1, wherein R² comprises —CH₂—CH₂—O—CH₂—CH₂—O—.

7. The compound of claim 1, wherein R² is

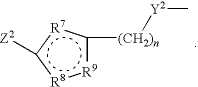

8. The compound of claim 7, wherein R² is: 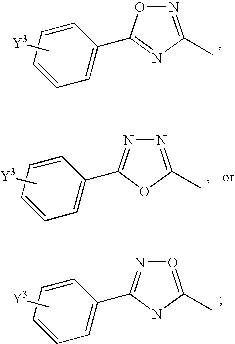
where Y³ is (CH₃)₃C—, CH₃CH₂(CH₃)₂C—, CH₃CH₂CH₂—, CH₃(CH₂)₂CH₂—, CH₃(CH₂)₄—CH₂—, (CH₃)₂CHCH₂—, (CH₃)₃CCH₂—, CH₃CH₂O—, (CH₃)₂CHO—, or CF₃CH₂CH₂— or a group having the formula:

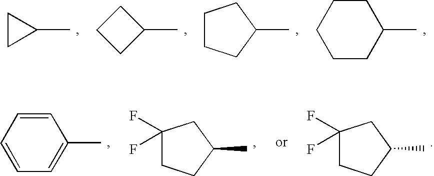

9. The compound of claim 8, wherein R² is:

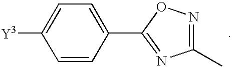

10. The compound of claim 9, wherein R² is:

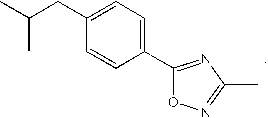

11. The compound of claim 7, wherein R² is:

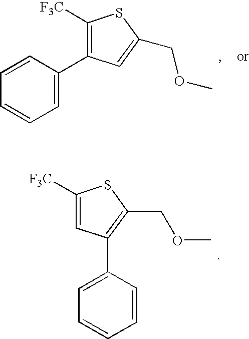

12. The compound of claim 11, wherein R² is

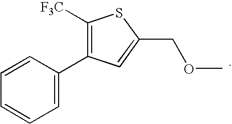

13. The compound of claim 1, wherein R² has formula IV

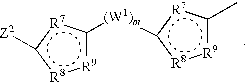

14. The compound of claim 13, wherein R² is

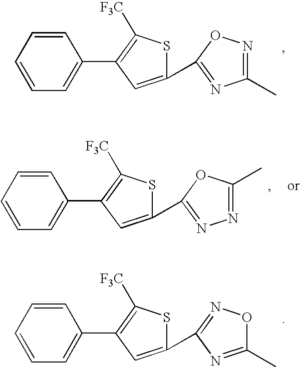

15. The compound of claim 1, wherein R² is (C₁-C₁₀)alkyl, (C₂-C₁₀)alkenyl and (C₂-C₁₄)alkynyl, (C₁-C₁₀)alkoxy or (C₂-C₁₆)alkoxyalkyl.

16. The compound of claim 15, wherein R² is (C₁-C₁₀)alkyl, (C₁-C₁₀)alkoxy or (C₂-C₁₂)alkoxyalkyl.

17. The compound of claim 16, wherein R² is methyl, ethyl, propyl, butyl, pentyl, hexyl, heptyl, octyl, trifluoromethyl, trifluoroethyl, trifluoromethoxy, trifluoroethoxy, methoxy, ethoxy, propoxy, butoxy, pentoxy, heptoxy, or octoxy.

18. The compound of claim 1, wherein each of X¹, Y¹ and Z¹ is CH₂.

19. The compound of claim 1, wherein R³ is hydrogen, methyl, hydroxymethyl, ethyl, hydroxyethyl, propyl, or isopropyl.

20. The compound of claim 19, wherein R³ is hydrogen, methyl, hydroxymethyl, ethyl, or hydroxyethyl.

21. The compound of claim 1, having the formula

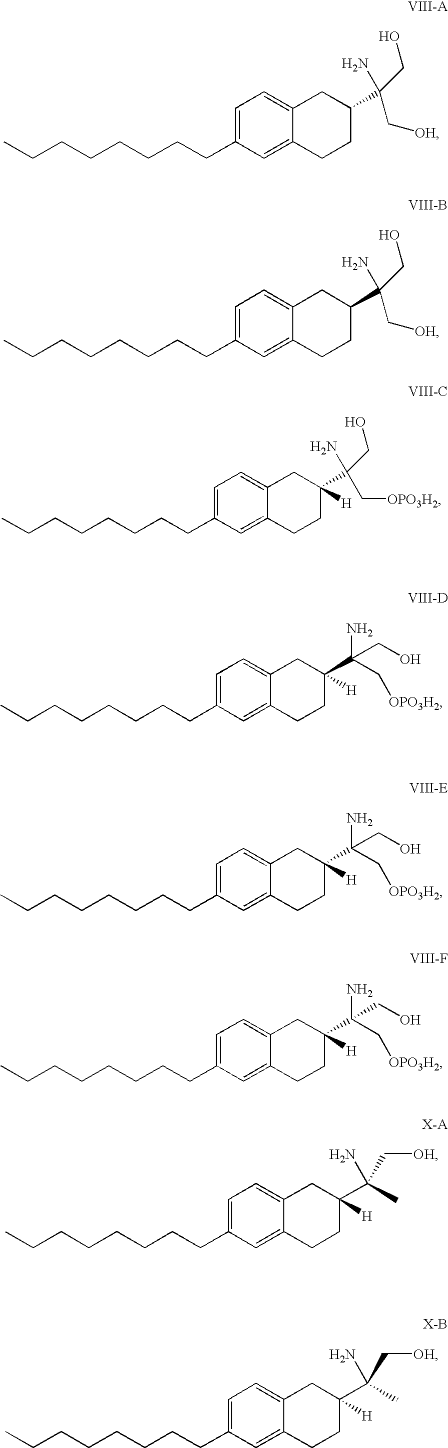

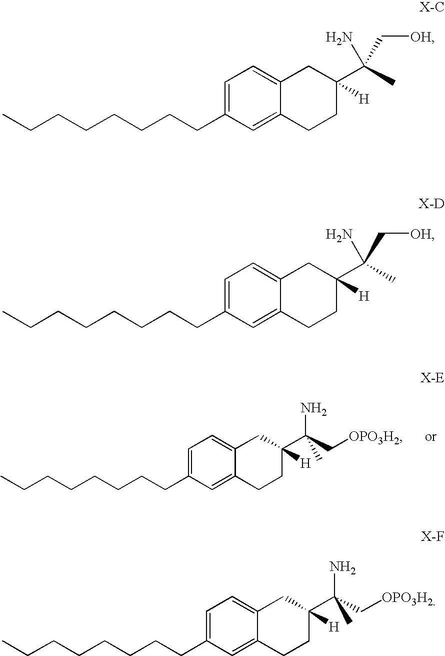

22. The compound of claim 1, having an enantiomeric excess of at least 90%.

23. A pharmaceutical composition comprising an effective amount of an enantiomerically pure compound of formula I of claim 1.

24. A method for preparing synthesizing an enantiomer of a compound of claim 1 comprising the step of separating the isomers of a compound of the formula 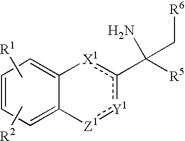
wherein R⁵ is hydrogen, (C₁-C₁₀)alkyl, hydroxy(C₁-C₁₀)alkyl, (C₁-C₁₀)alkoxy or —CO₂R^{d};
R⁶ is hydroxyl (—OH) or —CO₂R^{d};
X¹, Y¹ and Z¹ are independently O, CR^{a}, CR^{a}R^{b}, N, NR^{c}, or S;
R¹ and R² are independently hydrogen, halo, halo(C₁-C₁₀)alkyl, cyano, —NR^{a}R^{b}, (C₁-C₂₀)alkyl, (C₂-C₂₀)alkenyl, (C₂-C₂₀)alkynyl, (C₁-C₂₀)alkoxy, (C2-C₂₆)alkoxyalkyl, (C₃-C₁₂)cycloalkyl, (C₆-C₁₀)aryl, (C₇-C₃₀)arylalkyl, (C₂-C₁₀)heterocyclic, (C₄-C₁₀)heteroaryl, or (C₄-C₁₀)heteroaryl(C₁-C₂₀)alkyl; or
R² can be a group having formula II, III, IV, V, or VI; 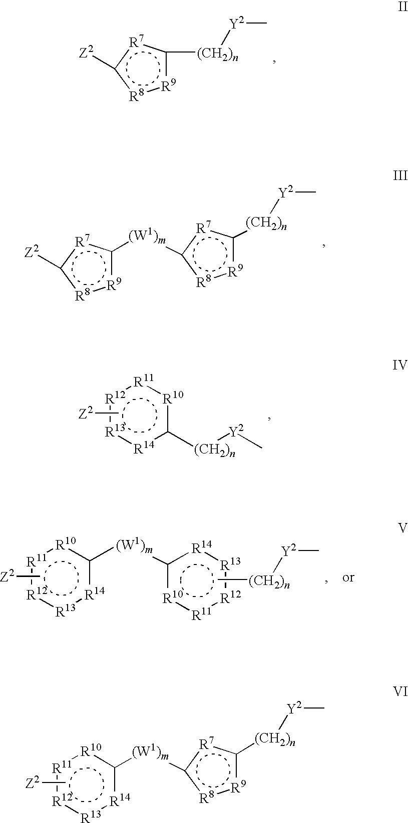
R⁷, R⁸, R⁹, R¹⁰, R¹¹, R¹², R¹³, and R¹⁴ are independently O, S, C, CR¹⁵, CR¹⁶R¹⁷, C═O, N or NR¹⁸;
R¹⁵, R¹⁶ and R¹⁷ are independently hydrogen, halo, (C₁-C₁₀)alkyl, (C₁-C₁₀)alkyl substituted with halo, hydroxy, (C₁-C₁₀)alkoxy, or cyano; and where R¹⁸ can be hydrogen or (C₁-C₁₀)alkyl;
where Z² is hydrogen, halo, halo(C₁-C₁₀)alkyl, cyano, —NR^{a}R^{b}, (C₁-C₂₀)alkyl, (C₂-C₂₀)alkenyl, (C₂-C₂₀)alkynyl, (C₁-C₂₀)alkoxy, (C₂-C₂₆)alkoxyalkyl, (C₃-C₁₂)cycloalkyl, (C₆-C₁₀)aryl, (C₇-C₃₀)arylalkyl, (C₂-C₁₀)heterocyclic, (C₄-C₁₀)heteroaryl, or (C₄-C₁₀)heteroaryl(C₁-C₂₀)alkyl; wherein the alkyl, alkenyl, alkynyl, cycloalkyl, aryl, heterocyclic, or heteroaryl groups of Z² are optionally perfluorinated or optionally substituted with 1, 2, 3, or 4 groups where the substituent groups are independently hydroxy, halo, cyano, (C₁-C₁₀)alkoxy, C₆-aryl, (C₇-C₂₄)arylalkyl, oxo (═O), or imino (═NR^{d}), wherein one or more of the carbon atoms in the Z² alkyl groups can be independently replaced with non-peroxide oxygen, sulfur or NR^{c}; [symbol]
indicates one or more optional double bonds;
wherein Y² is a bond, O, S, C═O, or NR^{c}, CH₂; W¹ is a bond; —CH₂— and m is 1, 2, or 3, or (C═O)(CH₂)₁₋₅ and m is 1; wherein W¹ is optionally interrupted with non-peroxide O, S, C═O, or NR^{c};
each [symbol] represents an optional double bond; and n is 0, 1, 2, or 3;
R^{a}, R^{b}, R^{c} and R^{d} are independently hydrogen, or (C₁-C₁₀)alkyl;
wherein the
alkyl, alkenyl, alkynyl, cycloalkyl, aryl, heterocyclic, or heteroaryl groups of R¹ and R² independently are optionally perfluorinated or optionally substituted with 1, 2, 3, or 4 groups where the substituent groups are independently hydroxy, halo, cyano, (C₁-C₁₀)alkoxy, C₆-aryl, (C₇-C₂₄)arylalkyl, oxo (═O), or imino (═NR^{d}), wherein one or more of the carbon atoms in the R¹ or R² alkyl groups can be independently replaced with non-peroxide oxygen, sulfur or NR^{c}; the alkyl groups of R³ are optionally substituted with 1, or 2 hydroxy groups; and R^{d} is hydrogen, or (C₁-C₁₀)alkyl to obtain the enantiomerically pure isomer; and
transforming the isomer into a compound having formula (I).

25. A method for prevention or treatment of a pathological condition or symptom in a mammal, wherein the activity of sphingosine 1-phosphate receptors is implicated and agonism of such activity is desired, comprising administering to said mammal an effective amount of a compound of claim 1.

26. The method of claim 25, wherein the pathological condition is an autoimmune disease.

27. The method of claim 26, wherein the autoimmune disease is uveitis, type I diabetes, rheumatoid arthritis, inflammatory bowel diseases, or multiple sclerosis.

28. The method of claim 27, wherein the autoimmune disease is multiple sclerosis.

29. The method of claim 28, wherein the prevention or treatment of the pathological condition is altering lymphocyte trafficking.

30. The method of claim 29, wherein altering lymphocyte trafficking provides prolonged allograft survival.

31. The method of claim 30, wherein the allograft is for transplantation.

32. A method for prevention or treatment of a pathological condition or symptom in a mammal, wherein the activity S1P lyase implicated and inhibition of the S1P lyase is desired, comprising administering to said mammal an effective amount of a compound of claim 1.
